(19) United States
(12) Patent Application Publication
(10) Pub. No.: US 2015/0110802 A1
(43) Pub. Date: Apr. 23, 2015

(54) ANTIBODY PRODUCTION METHODS

(71) Applicant: MedImmune, LLC, Gaithersburg, MD (US)

(72) Inventors: Partha S. Chowdhury, Gaithersburg, MD (US); Chew-Shun Chang, Gaithersburg, MD (US)

(73) Assignee: Medlmmune, LLC, Gaithersburg, MD (US)

(21) Appl. No.: 14/380,128

(22) PCT Filed: Feb. 26, 2013
(86) PCT No.: PCT/US2013/027785

§ 371 Date: Aug. 21, 2014

Related U.S. Application Data
(60) Provisional application No. 61/604,582, filed on Feb. 29, 2012.

(51) Int. Cl. C07K 16/40; A61K 39/02; A61K 39/085; C07K 16/12; C07K 16/22; C07K 16/28; A61K 39/00

(52) U.S. Cl. CPC C07K 16/40; C07K 16/2863; C07K 16/2803; A61K 39/02; A61K 39/0005; C07K 16/1271; C07K 16/12; C07K 16/22; A61K 39/085; A61K 2039/70; C07K 2317/10
USPC 424/145.1; 424/158.1; 424/236.1; 424/198.1; 424/146.1; 436/547; 436/548

(57) ABSTRACT

Methods are provided for producing a plurality of antibody species in a single animal, comprising delivering a plurality of antigen species to a single animal, where each antigen species is delivered to the animal at an anatomically distinct location. Also provided are methods for generating an immune response in an animal, where each antigen species is delivered to the animal at an anatomically distinct location, under conditions in which the animal produces a plurality of antibody species, where each antibody species specifically binds to a different antigen species among the plurality of antigen species.

### CROSS REFERENCE TO RELATED APPLICATIONS

This application claims priority to U.S. Provisional Application No. 61/604,582 filed Feb. 29, 2012, which is incorporated by reference in its entirety.

### FIELD

The technology relates in part to the production of antibodies.

### BACKGROUND

Antibodies, which also are referred to as immunoglobulins (Ig), are proteins that naturally occur in blood or other bodily fluids of vertebrates. Antibodies are immune system agents that bind to and neutralize foreign objects, such as bacteria and viruses.

Antibodies may be generated in animals upon exposure to one or more antigens. Antibody preparations can be derived from immunized animals and can include monoclonal and polyclonal preparations. Monoclonal antibodies are highly specific, being directed against a single antigenic site, whereas polyclonal antibody preparations can include different antibodies directed against different antigens or antigenic sites. Such antibody preparations can be useful for a variety of applications including laboratory assays, diagnostics and therapeutics.

### SUMMARY

Provided herein are methods for producing a plurality of antibody species in a single animal, comprising delivering a plurality of antigen species to a single animal, where each antigen species is delivered to the animal at an anatomically distinct location, under conditions in which the animal produces a plurality of antibody species, where each antibody species specifically binds to a different antigen species among the plurality of antigen species.

Also provided are methods for generating an immune response, comprising delivering a plurality of antigen species to a single animal, where each antigen species is delivered to the animal at an anatomically distinct location, under conditions in which the animal produces a plurality of antibody species, where each antibody species specifically binds to a different antigen species among the plurality of antigen species.

Also provided are methods for producing a plurality of monoclonal antibody species, comprising (a) generating a plurality of hybridoma species from cells from an animal to which a plurality of antigen species has been delivered, where each antigen species is delivered to the animal at an anatomically distinct location; and (b) isolating a plurality of monoclonal antibody species from the hybridomas, where each antibody species specifically binds to a different antigen species among the plurality of antigen species.

Certain aspects are described further in the following description, examples, claims and drawings.

### BRIEF DESCRIPTION OF THE DRAWINGS

The drawings illustrate aspects of the technology and are not limiting. For clarity and ease of illustration, the drawings are not made to scale and, in some instances, various aspects may be shown exaggerated or enlarged to facilitate an understanding of particular aspects.

FIG. 1. Panels A and B show schematic of several different representative sites where different antigens may be injected. Panel A shows site A (axillary draining lymph nodes), site B (inguinal lymph nodes) and site C (popliteal). To locate each lymph node draining site, animal is spatially divided into three zones (top, mid and lower section). The axillary draining lymph node sites are located close to the arm pits of the upper limbs. The inguinal draining lymph nodes are located close to the mid-section of the animal. The popliteal lymph node draining sites are located behind the hind legs. As shown in panel B, the animal may be further divided into two halves (left, and right) thus sub-dividing each to provide sites A1 and A2 (left and right axillary draining lymph nodes, collectively referred to as site A), sites B1 and B2 (left and right inguinal lymph nodes, collectively referred to as site B) and sites C1 and C2 (left and right popliteal, collectively referred to as site C). (Adapted from Kilpatrick et al. 1997, HYBRIDOMA, 16:381-9). The location of specific lymph nodes within the mouse is provided in panel C, As taught herein different antigens are delivered to anatomically distinct locations which may include intranoda delivery to different lymphnodes and/or intrasplenic delivery,

FIG. 2. Serum samples from mice prior to immunization (pre-bleeds) were analyzed by ELISA for the presence of antibodies binding to any of the antigens. No reactivity to huRAGE-his (top left plot), huHer3-his (top right plot), α-toxin-his (bottom left plot) or a control antigen not used for immunization (gp130, bottom right plot) was seen in any of the samples. Representative data from animals 9102, 9104, 9105, 9109, 9110 and 9111 are shown.

FIG. 3. Serum samples from mice post immunization (post-bleeds) were analyzed by ELISA for presence of antigen reactive antibodies. The post-bleed samples show high titers for huRAGE-his (injected at site A, top left plot); moderate titers to huHer3-his (injected at site B, top right plot); little to no titer to α-toxin-his (injected at site C, bottom left plot); or the control antigen (gp130 not injected, bottom right plot). The relative titers were as would be predicted for each of the three antigens.

FIG. 4. Anti-huRAGE-his titers (top left plot) from mice immunized with huRAGE-his alone (animals 9112 and 9113) were equivalent to those from mice which had been immunized with three antigens, each delivered to a different anatomical location (compare huRAGE plot, FIG. 3) indicating that multiple immunization does not appear to alter the immune response that can be generated. The anti-huHer3-his (top right panel plot), anti-α-toxin-his (bottom left plot), and anti-gp130 (bottom right plot) titers were near background levels.

FIG. 5. Anti-Her3-his titers (top right plot) from mice immunized with Her3-his alone (mouse 9121) were equivalent to those from mice which had been immunized with all three antigens, each delivered to a different anatomical location (compare Her3 μlot, FIG. 3) indicating that multiple immunization does not appear to alter the immune response that can be generated. Animal 9119 was inadvertently immunized once with huRAGE-his (in additional to Her3) in one of the dosing procedure. This leads to both anti-huRAGE-his (top left plot) and anti-Her3-his (top right plot) responses in this animal, while animal 9121 only shows a response to Her3-his. The anti-α-toxin (bottom left plot), and anti-gp130 (bottom right plot) titers were near background levels in both animals.

FIG. 6. Characterization of hybridomas obtained from lymph nodes and spleens of animals immunized with all three antigens. A very high percentage, ˜65%, of the hybridomas generated from the lymph nodes of site A (huRAGE injection) are positive for anti-huRAGE antibodies, only 2% were positive for anti-Her3 antibodies and no anti-α-toxin antibodies were detected (top left plot). A moderate percent of hybridomas generated from the lymph nodes of site B (Her3 injection) were positive for anti-Her3 antibodies, none were positive for anti-huRAGE antibodies and only 1 was positive for anti-α-toxin (top right plot). For hybridomas derived from lymph node of site C, no anti-α-toxin specific hybridomas were detected, as the animals mount no response to α-toxin (as indicated by the very low anti-α-toxin titers detected in the serum). However, hybridomas specific for the immunogenic antigens, huRAGE and huHer3, were detected (bottom left plot). A low percentage of hybridomas positive for any of the three antigens (˜11% anti-huRAGE; ˜2% anti-Her3 and no anti-α-toxin) were obtained from spleens (bottom right plot).

FIG. 7. Serum samples from mice prior to immunization (pre-bleeds) were analyzed by ELISA for the presence of antibodies binding to any of the antigens. No reactivity to mVEGF-his (top left plot), cynoKDR-his (top right plot), IsdB-his (bottom left plot) or a control antigen (mHer3-his, bottom right plot) was seen in any of the samples. Representative data from animals 4043, 4044, 4045, 4046 and 4047 are shown.

FIG. 8. Serum samples from mice post immunization (post-bleeds) were analyzed by ELISA for presence of antigen reactive antibodies. The post-bleed samples show high titers for IsdB-His (injected at site C, bottom left plot); moderate titers to cynoKDR-his his (injected at site B, top right plot); little to no titer to mVEGF-his his (injected at site A, top left plot); or the control antigen (mHer3-his, bottom right plot). The relative titers were as would be predicted for each of the three antigens.

FIG. 9. Anti-IsdB-his titers (bottom left plot) from mice immunized with IsdB-his alone (animals 5362 & 5363) were equivalent to those from mice which had been immunized with three antigens (animals 4043, 4044, 4045, 4046 and 4047), each delivered to a different anatomical location (IsdB plot, FIG. 8) indicating that multiple immunization does not appear to alter the immune response that can be generated. The higher background signal seen for the other antigens is likely due to an anti-his-tag response.

FIG. 10. Anti-cynoKDR-his titers (top right plot) from mice immunized with cynoKDR-his alone (mouse 5359 and 5360) were equivalent to those from mice which had been immunized with all three antigens (animals 4043, 4044, 4045, 4046 and 4047), each delivered to a different anatomical location (cynoKDR plot, FIG. 8) indicating that multiple immunization does not appear to alter the immune response that can be generated. The anti-mVEGF-his (top left plot), anti-IsdB-his (bottom left plot) and anti-mHer3-his (bottom right panel) titers were near background levels.

FIG. 11. Characterization of hybridomas obtained from lymph nodes and spleens of animals immunized with all three antigens. A high percentage, ˜30%, of the hybridomas generated from the lymph nodes of site C (IsdB-his injection) are positive for anti-IsdB antibodies, no anti-cynoDR or anti-mVEGF antibodies were detected (top left plot). Similar high percentage of hybridomas, ˜45%, generated from the lymph nodes of site B (cynoKDR injection) were positive for anti-cynoKDR antibodies, none were positive for anti-mVEGF antibodies and ˜2% was positive for anti-IsdB (top right panel). For hybridomas derived from site A, no anti-mVEGF hybridomas was detected. A very low percentage of anti-cynoKDR and anti-IsdB, ˜2%, were detected in hybridomas derived from site A (bottom left plot). A low percentage of hybridomas positive for any of the three antigens (˜2% anti-cynoKDR; no anti-mVEGF and anti-IsdB) were obtained from spleens (lower right plot).

### DETAILED DESCRIPTION

Using animals to generate antibodies against a collection of antigens can be a time and labor-intensive process. Often, a separate animal is used to generate antibodies against each type of antigen. Production of multiple antibodies using a single animal can be a more efficient approach. Immunization of an animal with a mixture of antigens, however, typically leads to different antigens competing with each other for the animal's immune resources. The immune response that follows often is generated against the antigen with the greatest immunogenicity, resulting in the production of antibodies to the most immunogenic antigen and not the others. Described herein are antibody production methods which involve delivering several antigens to an animal such that the antigens are physically isolated from one another and do not compete with each other for the same immune resource. Thus, provided herein are methods for producing multiple antibodies having different specificities in a single animal by delivering multiple antigens to the animal, each at a distinct anatomic location.

### Terminology

Methods provided herein often are not limited to specific compositions or process steps, as such may vary. Also, as used herein, the singular form “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or embodiments of the methods herein, which can be had by reference to the specification as a whole. Amino acids often are referred to herein by commonly known three letter symbols or by the one-letter symbols recommended by the IUPAC-IUB Biochemical Nomenclature Commission. Nucleotides, likewise, often are referred to by commonly accepted single-letter codes. Accordingly, the terms defined immediately below are more fully defined by reference to the specification in its entirety.

### Antibodies

An antibody is an immunoglobulin molecule that recognizes and specifically binds to a target, such as a protein, polypeptide, peptide, carbohydrate, polynucleotide, lipid, other haptens, or combinations of the foregoing through at least one antigen recognition site within the variable region of the immunoglobulin molecule. As used herein, the term “specifically binds” refers to an interaction between an antibody and a target such that the binding affinity of the antibody to the target is greater than the binding affinity of the antibody to a non-target. As used herein, the terms “antibody” and “antibodies”, also known as immunoglobulins, encompass monoclonal antibodies (including full-length monoclonal antibodies), polyclonal antibodies, multispecific antibodies comprising at least two different epitope binding domains (e.g., bispecific antibodies), human antibodies, humanized antibodies, camelised antibodies, chimeric antibodies, fusion proteins comprising an antigen determination portion of an antibody, and any other modified immunoglobulin molecule comprising an antigen recognition site so long as the antibodies exhibit the desired biological activity. In particular, antibodies include immunoglobulin molecules and immunologically active fragments of immunoglobulin molecules, i.e., molecules that contain at least one antigen-binding site. Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., G1 m (f, z, a or x), G2m(n), G3m(g, b, or c), Am, Em, and Km(1, 2 or 3)). Antibodies may be derived from any mammal, including, but not limited to, humans, monkeys, ungulates (e.g., pigs, horses, cattle, sheep, goats, and the like), rodents (e.g., mice, rats, guinea pigs, hamsters, and the like), rabbits, ferrets, dogs, cats, and the like or other animals such as birds (e.g. chickens). Antibodies can be naked or conjugated to other molecules such as toxins, radioisotopes, and the like.

Thus, antibodies are immunological proteins that bind a specific antigen. In most mammals, including humans and mice, antibodies are constructed from paired heavy and light polypeptide chains. Each chain is made up of two distinct regions, referred to as the variable (Fv) and constant (Fc) regions. The light and heavy chain Fv regions contain the antigen binding determinants of the molecule and are responsible for binding the target antigen. The Fc regions define the class (or isotype) of antibody (IgG for example) and are responsible for binding a number of natural proteins to elicit important biochemical events.

Each chain includes constant regions that are representative of the antibody class and variable regions specific to each antibody. The constant region determines the mechanism used to destroy antigen. Antibodies are divided into five major classes, IgM, IgG, IgA, IgD, and IgE, based on their constant region structure and immune function. The variable and constant regions of both the light and the heavy chains are structurally folded into functional units called domains. Each light chain consists of one variable domain (VL) at one end and one constant domain (CL) at its other end. Each heavy chain has at one end a variable domain (VH) followed by three or four constant domains (CH1, CH2, CH3, CH4).

An antibody generally is a Y-shaped protein. The arms of the Y contain the site that binds antigen and are called the Fab (fragment, antigen binding) region. Each Fab region is composed of one constant and one variable domain from each heavy and light chain of the antibody. The constant domain of the light chain is aligned with the first constant domain of the heavy chain, and the light chain variable domain is aligned with the variable domain of the heavy chain.

In some aspects, monoclonal antibodies are generated using the methods described herein. The term “monoclonal antibody” as used herein refers to an antibody obtained from a population of substantially homogeneous or isolated antibodies, e.g., the individual antibodies comprising the population are identical except for possible naturally occurring mutations that may be present in minor amounts. A monoclonal antibody is generally derived from a single clone, including without limitation, any eukaryotic, prokaryotic or phage clone, and may be a naturally occurring antibody (e.g. an antibody produced by a hybridoma clone) or a recombinant antibody (e.g., an engineered antibody produced in a transfected host cell), Monoclonal antibodies are highly specific, being directed against a single antigenic site or multiple antigenic sites in the case of multispecific engineered antibodies. Furthermore, in contrast to polyclonal antibody preparations which include different antibodies directed against different determinants (epitopes) and/or antigens, each monoclonal antibody is directed against the same determinant on an antigen. In addition to their specificity, monoclonal antibodies can be advantageous in that they may be synthesized uncontaminated by other antibodies. The modifier “monoclonal” is not to be construed as requiring production of the antibody by any particular method. Certain methods for the production of monoclonal antibodies are described in detail below.

### Antigens

In some aspects, a plurality of antigens is delivered to an animal. The term “antigen” as used herein refers to a molecule that causes an immune response when introduced into an organism and that is capable of binding to specific antibodies. Antibody-antigen binding is mediated by the sum of many interactions between the antigen and antibody including, for example, hydrogen bonds, van der Waals forces, and ionic and/or hydrophobic interactions. An antigen binds to the complementarity regions on an antibody. The corresponding region(s) of the antigen is referred to as an “antigenic determinant” or “epitope”. It is contemplated that one or more isolated antigenic determinant regions may be used as an antigen to generate an immune response directed to particular portions of an larger molecule. For example, an extracellular domain of a protein, or a peptide comprising at least a portion of a catalytic domain, are useful to generate antibodies which bind to these specific portions of the protein from which they are derived. Antigens include molecules such as, for example, polypeptides, polynucleotides, carbohydrates, haptens, and the like, from sources such as, for example, plants, animals, viruses, microorganisms, and the like. Antigens also can include substances such as toxins, chemicals, drugs, foreign particles, and the like.

In some aspects, antigens can include growth factors, cytokines, cytokine-related proteins, and receptors selected from among, for example, BMP1, BMP2, BMP3B (GDF10), BMP4, BMP6, BMP8, CSF1(M-CSF), CSF2 (GM-CSF), CSF3 (G-CSF), EPO, FGF1 (aFGF), FGF2 (bFGF), FGF3 (int-2), FGF4 (HST), FGF5, FGF6 (HST-2), FGF7 (KGF), FGF9, FGF10, FGF11, FGF12, FGF12B, FGF14, FGF16, FGF17, FGF19, FGF20, FGF21, FGF23, FGFR, FGFR1, FGFR2, FGFR3, FGFR4, FGFRL1, FGFR6, IGF1, IGF2, IGF1R, IGF2R, IFNA1, IFNA2, IFNA4, IFNA5, IFNA6, IFNA7, IFNAR1, IFNAR2, IFNB1, IFNG, IFNW1, FIL1, FIL1 (EPSILON), FIL1 (ZETA), IL1A, IL1B, IL2, IL3, IL4, IL5, IL6, IL7, IL8, IL9, IL10, IL11, IL12A, IL12B, IL13, IL14, IL15, IL16, IL17, IL17B, IL18, IL19, IL20, IL22, IL23, IL24, IL25, IL26, IL27, IL28A, IL28B, IL29, IL30, IL2RA, IL1R1, IL1R2, IL1RL1, IL1RL2, IL2RA, IL2RB, IL2RG, IL3RA, IL4R, IL5RA, IL6R, IL7R, IL8RA, IL8RB, IL9R, IL10RA, IL10RB, IL11RA, IL12RB1, IL12RB2, IL13RA1, IL13RA2, IL15RA, IL17R, IL17RA, IL17RB, IL17RC, IL17RD, IL18R1, IL20RA, IL20RB, IL21R, IL22R, IL22RA1, IL23R, IL27RA, IL28RA, PDGFA, PDGFB, PDGFRA, PDGFRB, TGFA, TGFB1, TGFB2, TGFB3, TGFBR1, TGFBR2, TGFBR3, ACVRL1, GFRA1, LTA (TNF-beta), LTB, TNF (TNF-alpha), TNFSF4 (OX40 ligand), TNFSF5 (CD40 ligand), TNFSF6 (FasL), TNFSF7 (CD27 ligand), TNFSF8 (CD30 ligand), TNFSF9 (4-1BB ligand), TNFSF10 (TRAIL), TNFSF11 (TRANCE), TNFSF12 (APO3L), TNFSF13 (April), TNFSF13B, TNFSF14 (HVEM-L), TNFSF15 (VEGI), TNFSF18, TNFRSF1A, TNFRSF1B, TNFRSF10A (Trail-receptor), TNFRSF10B (Trail-receptor 2), TNFRSF10C (Trail-receptor 3), TNFRSF10D (Trail-receptor 4), FIGF (VEGFD), VEGF, VEGFB, VEGFC, KDR, FLT1, FLT4, NRP1, IL1HY1, IL1RAP, IL1RAPL1, IL1RAPL2, IL1RN, IL6ST, IL18BP, IL18RAP, IL22RA2, AIF1, HGF, LEP (leptin), PTN, ALK and THPO.

In some aspects, antigens can include chemokines, chemokine receptors, and chemokine-related proteins selected from among, for example, CCL1(I-309), CCL2 (MCP-1/MCAF), CCL3 (MIP-1a), CCL4 (MIP-1b), CCL5 (RANTES), CCL7 (MCP-3), CCL8 (mcp-2), CCL11 (eotaxin), CCL13 (MCP-4), CCL15 (MIP-1d), CCL16 (HCC-4), CCL17 (TARC), CCL18 (PARC), CCL19 (MIP-3b), CCL20 (MIP-3a), CCL21 (SLC/exodus-2), CCL22 (MDC/STC-1), CCL23 (MPIF-1), CCL24 (MPIF-2/eotaxin-2), CCL25 (TECK), CCL26 (eotaxin-3), CCL27 (CTACK/ILC), CCL28, CXCL1(GRO1), CXCL2 (GRO2), CXCL3 (GRO3), CXCL5 (ENA-78), CXCL6 (GCP-2), CXCL9 (MIG), CXCL10 (IP 10), CXCL11 (1-TAC), CXCL12 (SDF1), CXCL13, CXCL14, CXCL16, PF4 (CXCL4), PPBP (CXCL7), CX3CL1 (SCYD1), SCYE1, XCL1 (Iymphotactin), XCL2 (SCM-1b), BLR1 (MDR15), CCBP2 (D6/JAB61), CCR1 (CKR1/HM145), CCR2 (mcp-1RB/RA), CCR3 (CKR3/CMKBR3), CCR4, CCR5 (CMKBR5/ChemR13), CCR6 (CMKBR6/CKR-L3/STRL22/DRY6), CCR7 (CKR7/EBI1), CCR8 (CMKBR8/TER1/CKR-L1), CCR9 (GPR-9-6), CCRL1 (VSHK1), CCRL2 (L-CCR), XCR1 (GPR5/CCXCR1), CMKLR1, CMKOR1 (RDC1), CX3CR1 (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Ra), IL8RB (IL8Rb), LTB4R (GPR16), TCP10, CKLFSF2, CKLFSF3, CKLFSF4, CKLFSF5, CKLFSF6, CKLFSF7, CKLFSF8, BDNF, C5R1, CSF3, GRCC10 (C10), EPO, FY (DARC), GDF5, HIF1A, IL8, PRL, RGS3, RGS13, SDF2, SLIT2, TLR2, TLR4, TREM1, TREM2, and VHL.

In some aspects, antigens can include cell surface proteins selected from among, for example, integral membrane proteins including ion channels, ion pumps, G-protein coupled receptors, structural proteins, adhesion proteins such as integrins, transporters, membrane-bound enzymes, proteins involved in accumulation and transduction of energy and lipid-anchored proteins including G proteins and some membrane-anchored kinases. In some aspects, antigens can include enzymes such as kinases, proteases, lipases, phosphatases, fatty acid synthetases, digestive enzymes such as pepsin, trypsin, and chymotrypsin, lysozyme, and polymerases. In some aspects, the antigens include receptors such as hormone receptors, lymphokine receptors, monokine receptors, growth factor receptors, G-protein coupled receptors, and the like.

Additional examples of antigens that can be used with the methods provided herein include, without limitation, 14-3-3 Sigma, 2F7, 6B9, 8-oxoguanine DNA glycosylase, Abdominal-B, ABP2 olfactory binding protein, Abrupt, acetylcholine nicotinic receptors (e.g., neuronal), acetylcholine nicotinic receptors (e.g., muscle), acetylcholinesterase, Achaete protein, acinar (e.g., exocrine gland), Acj6, actin, actin associated antigen (e.g., migration-related), actin binding protein 34 (ABP34), alpha-actinin, actinin (e.g., smooth muscle alpha), addressin (PNAd), adducin-related protein, adenovirus type 5 hexon, agrin, aldo-keto reductase family 1 member B1, aldo-keto reductase family 1 member C2, alkaline phosphatase isoenzyme, alkaline phosphatase (e.g., bone and liver), allatostatin, alpha PKA-R, alpha sarcoglycan, alpha-2-macroglobulin receptor, alpha-L-fucosidase, Akt1/PKB, aminopeptidase N, angiotensin converting enzyme, anion exchanger 1, annexin I, annexin II, Antennapedia protein, AP-2 alpha, AP-3 (e.g., delta subunit), APA2, APEX nuclease I, apoptotic marker in phagocytic cells, argos-gene product, Armadillo Drosophila protein, Aspergillus flavus isolate 93803, ATPase (e.g., Na(+) K(+) alpha subunit), ATPase (e.g., Na(+) K(+) alpha-1 subunit), ATPase (e.g., Na(+) K(+) beta-1 subunit), ATPase (e.g., Na(+) K(+) beta-subunit), ATPase (e.g., Ca(+2) fast twitch SR), ATPase (e.g., Ca(+2) slow twitch/cardiac SR), autoimmune double stranded DNA, autoimmune single stranded DNA, avian myoblastosis virus (p19̂), axonal filaments (e.g., 56 & 58 kDa), axons (CNS), baculovirus, bam (Fly Bag-of-marbles), basement membrane marker, BCL2-like 1, BCL2-like 2, BEAF, beta-defensin 3, beta-galactosidase, Bicaudal-D, blastemal regeneration cell marker of the newt, Blood Group A, Blood Group A, Blood Group A1B, Blood Group ABH, Blood Group B, Blood Group Lewis a, Blood Group Lewis b, Sialyl Lewis a, bodywall muscle cells, bone sialoprotein II, Botrytis cinerea, BrdU, Broad (core), Broad (Z1), Broad (Z3), bruchpilot, BVES, c-myc, cactus, cadherin-6B, cadherin-7, cadherin-8, cadherin, B-cadherin, C-cadherin, DE-cadherin, DN-cadherin, E-cadherin, N-cadherin, R-cadherin, calbindin-32, calcyclin (prolactin receptor associated protein), calmodulin, Calnexin, calreticulin (recombinant Dictyostelium), Cap32/34, capping protein alpha-1 & alpha-2 subunits, capping protein beta-1 subunit, capping protein beta-2 subunit, carbohydrate epitope, carbonic anhydrase VIII, cardioactive peptide B, CARO2, catenin (alpha N), alpha-catenin, beta-catenin, catenin, catenin (p120), CAV1, Cbl-L protein exon 6 (e.g., aa 449-878 of L isoform), CD1-1, CD1a, CD2 (LFA-2), CD3, CD4, CD5, CD6, CD7, CD8, CD9, CD10, CD11a (LFA-1 alpha subunit), CD11b (Mac-1 alpha subunit), CD11b (Mac-1, CR3), CD13, CD14, CD15, CD16, CD17, CD18, CD18 (beta subunit of CD11a, b, c), CD19, CD20, CD21, CD22, CD24, CD25, CD26, CD27, CD28, CD29, CD30, CD31, CD33, CD34, CD36, CD38, CD40, CD41, CD41a, CD41b, CD42b, CD43, CD44 (hyaluronate receptor), CD45 (lymphocyte common antigen), CD45R0, CD45RA, CD45RB, CD46, CD47, CD48, CD50, CD52, CD53, CD54, CD55, CD56, CD58 (LFA-3), CD59, CD62E, CD62L, CD62P, CD63 (LIMP), CD64, CD66, CD69, CD71, CD72, CD74, CD79a, CD80, CD81, CD83, CD84, CD86, CD90, CD95/Fas, CD97, CD98, CD99R, CD105, CD106, CD107a, CD107b, CD108, CD117, CD135, CD138, CD140a/PDGF-RA, CD140b/PDGF-RB, CD147, CD158d/KIR2DL4, CD162, CD163, CD177, CD180, CD203c, CD205, CD222, CD229, CD230/Human Prion Protein (PrP), CD235a, CD253/TRAIL, CD261/TRAIL-R1, CD262/TRAIL-R2, CD263/TRAIL-R3, CD264/TRAIL-R4, CD300a, CD324/E-Cadherin, CD326/EpCAM, CD326/EpCAM, CD340/ErbB2/HER2, CD358/DR6, CD361, Cdk1, CPNE7, Csk, cell surface carbohydrate, cell-junction marker, chaoptin (e.g., sensory neurons), chemokine (C—X—C motif) ligand 9, chicken cell marker, chloride intracellular channel 1, choline acetyltransferase, chondroitin sulfate proteoglycans (e.g., carbohydrate epitope), chromogranin A (parathyroid secretory protein 1), chromosome 1 (e.g., 55-73 kDa polypeptides), chromosome 11 (e.g., 40 and 80 kDa polypeptides), chromosome 12 (e.g., 21 kDa product), chromosome 19 (e.g., 35-69 kDa polypeptides), CNS-specific antigen, Coactosin p17, collagen (pro-) type I (e.g., aminopropeptide), collagen type II, collagen type III, collagen type IV, collagen type IX, collagen type VI, collagen type X, collagen type XII, collagen type XVIII, pro-collagen type I, collagenase, Connectin, connexin 32, Coracle, Coronin, Coronin N-terminal, Cortexillin I, Cortexillin II, Costal-2 (Cos2), chromosome 1 (e.g., 80 kDa polypeptide), creatine kinase, creatine kinase B chain, CrebA transcription factor, Crumbs Drosophila protein, CRYAB (crystallin alpha B), crystal protein, csA (contact site A glycoprotein), Csp, CSP2 chemosensory protein, Cubitus interruptus, Cutoff protein (CG3190, e.g., full length protein), Cut protein product, cyclic nucleotide-gated channel (rOCN2), cyclin A, cyclin B, CYP-33E1, cystatin A, cysteine string protein (CSP), cytokeratin 19, cytokeratin Endo-A, cytokeratin type II, Dacapo, Dachshund protein, Dally-like protein (Dip), DAO5, decorin, Delta (e.g., extracellular domain), desmin, dFMR1, dFMRP, DHPR alpha-subunit (e.g., skeletal muscle), DHPR beta-subunit (e.g., skeletal muscle), dipeptidylpeptidase IV, Disabled protein, Discoidin I (e.g., cAMP binding domain), discs large, DLAR, DMPK, DNA-damage-inducible transcript 3, dorsal, duct (e.g., exocrine gland), DYN-1, Dynein heavy chain, beta-dystroglycan, dystrophin, ecdysone receptor (EcR common), ecdysone receptor (EcR-A), ecdysone receptor (EcR-B1), egg shell marker for C. elegans embryos, elav Drosophila protein, Emerin (e.g., amino acids 11-17), Emerin (e.g., amino acids 69-77), Emerin (e.g., amino acids 7-15), Emerin (e.g., amino acids 89-96), Emerin (e.g., amino acids 112-115), Emerin (e.g., amino acids 112-115 and 150-158), Emerin (e.g., amino acids 152-159), Emerin (e.g., amino acids 221-228), enabled, endoglin, endoglin (CD105), endoplasmic reticulum (e.g., rough, glycoprotein), endothelial cell marker, endothelial cell surface, Engrail-1, engrailed/invected gene products, entactin (e.g., synaptic), Ep-CAM, EphB1, EphB2, EphB3, ephrin-B1, epidermal growth factor receptor, epithelial stem cell marker, epithelial surface marker (e.g., apical), EPS15, ER81, ERM-1, estrogen receptor alpha (e.g., ligand binding domain (aa 304-554)), even-skipped protein, Evx1, Extradenticle protein (EXDHDcc), Extra Sex Combs (ESC), eyeless protein (e.g., linker region), Eyes Absent (Eya) protein, Ezrin (p81), fasciclin I, fasciclin II, fasciclin III, fascin, fatty acid-binding protein (e.g., epidermal), fatty acid-binding protein (e.g., intestinal), fibrillin 2-like, fibronectin, fibronectin (e.g., cartilage specific V+C−), fibronectin (e.g., III-14 in Hep2), fibronectin (e.g., III-15), filament antigen, Fimbrin, Fimbrin (p67), Flamingo, floor plate marker, fluorescein, FMRP, FMRP (e.g., residues #1-204), Frizzled protein, Frizzled2, Fused (Fu), FXR1, FXR2, GD2 glycolipid, gelsolin, gemin4, gemin5, gemin6, geminin, germ cells, germ-line-specific P granules, gigas gene product, glass Drosophila protein, glial precursor, Glorund, glutamate-cysteine ligase regulatory subunit, glutamate receptor subunit (DgluR-IIA), glutamic acid decarboxylase, glutathione S-transferase Mu1, glutathione S-transferase Mu2, glutathione S-transferase Mu3, glycolipid (e.g., alpha-galactose lactoseries carbohydrate epitope), glycoprotein (e.g., 38 kDa, mucin-like), GOBP1 olfactory binding protein, GOBP2 olfactory binding protein, gp210, Groucho protein, GST-Klarsicht alpha protein (e.g., amino acids 1895-2262), GST-Klarsicht alpha protein (e.g., amino acids 277-556), GST-Klarsicht alpha protein (e.g., amino acids 875-1169), Gurken protein, Half pint protein (e.g., C-terminal half (aa 277-637)), HCP4, Headcase protein (e.g., aa 1-417), hematopoetic/neuronal cell surface glycoprotein, hemapoietic stem cell protein (e.g., 74 kDa), hemocytes, heparan sulfate proteoglycan, heparan sulfate proteoglycan (e.g., basement membrane), Heterochromatin Protein 1, hexokinase (e.g., Type I isozyme), highwire, hindsight protein, Hisactophilin (Dd gelation factor), HLA-A2, HLA-Class I, HLA-DQ1+DQ3, HLA-DR, HLA-DR+DP, HLA-DR1 (empty), HLA-E, HLA-G, HLFA (e.g., beta-subunit), HMR-1, HNF3b, Hoxb4, Hoxc10, Hoxc9, HSP-60, htsRC, huntingtin, hypodermal marker, hypodermis (e.g., seam cells), Ia antigen, ICAM-1, IL-8 (interleukin 18), pro-insulin (e.g., non-processed), pro-insulin (e.g., C-peptide), integrin alpha-5, integrin alpha-6, integrin alpha-7, integrin alphaPS1, integrin alphaPS2, integrin beta, integrin beta-1, integrin beta-3, integrin betaPS, integrin alpha-3, intercostal nerves, intercostal nerves in a rostrocaudal gradient, intermediate filament subunit, Islet-1 homeobox, Islet-1 specific homeobox, Islet-2, Jagged1, kel 1B, keratin sulfate, keratin sulfate (e.g., brain), keratin type I, keratinocyte (e.g., basal, cell attachment antigen), kinesin, kinesin-like protein KIF2C, L-CAM, L1 protein, L1-like CAM, lactadherin, lactase-phlorizin hydrolase, lactoylglutathione lyase, lacunin, lamin, lamin C, lamin DmO, laminin, laminin B2, laminin-binding lectin, S-laminin, laminin (fibronectin receptor), LAMP (gene symbol: Lsamp), LAMP-1, LAMP-1 (e.g., 110 kDa lysosomal membrane glycoprotein), LAMP-2, LAMP-2 (e.g., 110 kDa lysosomal membrane glycoprotein), late bloomer, LET413, leukocyte (e.g., activated, cell surface glycoprotein), Lim 1+2, Lim 3, link protein, LMP-1, LMX, lozenge, lysosomal membrane glycoprotein (cv24), lysosomal membrane glycoprotein (LEP-100), M2 (membrane protein), M6 (membrane protein), major sperm protein (MSP), maltase-glucoamylase, maltate dehydrogenase 1 NAD (e.g., soluble), mannose 6-phosphate/IGF II receptor (e.g., cation-independent), mannose 6-phosphate receptor (e.g., cation-dependent), Math1, melanoblasts and melanocytes (e.g., of neural crest origin), melanoma-associated antigen 4, meprin, mesoderm, metastasin 100 calcium binding protein A4 (calvasculin), methyl CpG binding protein 1, MHC class I, MHC class II, microfibrils, beta2-microglobulin, mineralocorticoid receptor, mitochondria, mitogen-activated protein kinase 14, Mmp1 catalytic domain, Mmp1 hemopexin domain, MNR2, moesin, MSX1+2, Muc4, muscle fast C-protein, muscle marker, muscle slow C-protein, muscle/neurite marker, Myeloperoxidase (MPO), myoblast marker, myoblast (chondrocyte) marker, myoblast (fibroblast) marker, myoblasts/myotubes (e.g., cell surface), MyoD, myogenin, myomesin, myosin (e.g., embryonic), myosin (e.g., all fibers), myosin (e.g., fast fibers), myosin (e.g., neonatal slow and fast lia fibers), myosin (e.g., slow fibers), myosin (e.g., neonatal and adult fast fibers), myosin (e.g., neonatal fast lia fibers), myosin fast muscle light chain 2, myosin heavy chain, myosin heavy chain 2A, myosin heavy chain 2B, myosin heavy chain A, adaxial, myosin heavy chain (e.g., all but 2X), myosin heavy chain (e.g., embryonic and adult fast), myosin heavy chain (e.g., embryonic and neonatal fast), myosin heavy chain (e.g., fast, 2A), myosin heavy chain (e.g., fast, 2B), myosin heavy chain (e.g., fast, 2X), myosin heavy chain (e.g., fast, extraocular specific), myosin heavy chain (e.g., fast, jaw muscle specific), myosin heavy chain (e.g., neonatal and adult), myosin heavy chain (e.g., neonatal fast), myosin heavy chain (e.g., sarcomere), myosin heavy chain (e.g., slow, alpha- and beta-myosin heavy chain, slow and 2A), myosin heavy chain (e.g., slow, SM1 only), myosin heavy chain (e.g., slow, SM2), myosin heavy chain (e.g., slow, SM2 only), myosin heavy chain (e.g., SM2 and atrial), myosin heavy chain (e.g., ventricular), myosin II, myosin IIB (e.g., cytoplasmic non-muscle), myosin II heavy chain, myosin light chain 1 and 3f (LC1f/3f), myosin light chain 1s (LC1s), myosin light chain 1s, 2s, 1f and 2f (LC1s, LC1f, LC2s, LC2f), myosin-Vila, myosin (e.g., sarcomere), myotactin, myotendinous antigen (tenascin), Na—K—Cl cotransporters, NAPA-73 (neurofilament-associated protein, e.g., 73 kDa), NCAM, NCAM (e.g., cytoplasmic domain), NCAM (e.g., extracellular domain), NCAM (e.g., sialylated form), NCAM/L1CAM leech homologue, Nervana protein, nervous system, nestin, neural associated ganglioside, neural crest cells, neural marker, neural precursor cells, neural retinal gangliosides (9-0-acetyl-GD3), neural specific, neural tube (e.g., dorsal), neurocan (e.g., C-terminal epitope), neurocan (e.g., N-terminal epitope), neurocan receptor, neurofilament (e.g., 160 kDa), neurofilament (e.g., 165 kDa), neurofilament-associated antigen, neurofilaments (e.g., primary sensory and motor), neurogenin 3, neuroglian, Neuromedin-B peptide, neuromuscular junction and reactive Schwann cell associated antigen, neuron (motor) antigen, neuronal cell surface marker, neuronal cell surface marker (e.g., SC-1, DM-GRASP, BEN), neuronal marker (e.g., cytoplasmic), neuronal marker (TAG-1), neuronal (e.g., mesencephalic trigeminal cell surface marker), neuronal (motor) marker (SC-1), neurons, neuropil region and primary motor neuron axons, Neuropilin-1, neurotactin, NFATc1, NFATc2, nidogen/entactin, Nkx2.2, NMES (nucleoside diphosphate kinase B), Notch (e.g., extracellular domain, EGF repeats #12-20), Notch (e.g., extracellular domain, EGF repeats #5-7), Notch (e.g., intracellular domain), Notch1, Notch2, notochord and neuropil, notochord marker, NrCAM, nuclear lamins II/III, nuclear membrane marker, nucleolar protein, nucleolin (e.g., 95 kDa and 90 kDa isoforms), nucleolin (e.g., 95 kDa isoform), nucleoplasmin, nullo-GST fusion protein (e.g., entire inframe nullo protein, GST at c-terminus), Numb, oligodendrocyte (myelin) marker, oligodendrocytes and their processes, optic nerve, orb protein, orb2 protein, ORC2, ornithine decarboxylase 1, Osa, osteonectin, otoferlin, Otx1, outer membrane protein-19 (OMP-1g), p46 cell surface protein excluded from macropinocytic, cup, p53, p80 endosomal membrane protein, PABA peptide hydrolase (meprin), PAR-3, PAS-7, paramyosin, Patched, Pax3, PAX6, PAX7, PBP2 olfactory binding protein, PBP3 olfactory binding protein, PDF, Pdx1, PDZ and LIM domain 1, peanut gene protein products, PECAM, PECAM-1, Peptide II B 80 kD, Pericardin, perlecan, perlecan (e.g., domain IV), peroxiredoxin 4, Pgp-1 (Ly-24) lymphocyte cell adhesion glycoprotein, P granule, P granule+body muscle, pharyngeal marker, phosphacan-KS, phosphacan/protein tyrosine phosphatase−z/b, phosphatidylinositol-specific phospholipase C, phosphoserine aminotransferase 1, phosphoserine phosphatase like, photoreceptors (e.g., rods and cones), photoreceptors (e.g., rods only), pigment cell marker, plateins (e.g., alpha-, beta- & gamma-), plateins (e.g., beta- & gamma-), Porphyromonas gingivalis, Pop1 (BYES), porin, Posterior sex combs protein, primordial germ cell surface marker, profilin, profilin II proteins, Prospero protein, proteasome 26S non-ATPase regulatory subunit 4, Proteasomen subunit 5, Proteasome subunit, protein tyrosine phosphatase (e.g., receptor-linked, DPTP10D), protein tyrosine phosphatase (e.g., receptor-linked, DPTP69D), protein tyrosine phosphatase (e.g., receptor-linked, DPTP99A), protein phospatase 2A, protein tyrosine phosphatase—z/b, proteoglycan (e.g., hyaluronic acid binding region), PTP-ER, quail cell marker, radial cells and radial glial cells (vimentin), radial glial cell marker, radial glial cells, ras-related C3 botulinum toxin substrate 1, Rb (e.g., last 200 amino acids (GST-Rb)), RecD protein, reelin, Relish, reovirus, Repo, retinal space (mechanoreceptors), rho1, rhoB, rhodopsin, RME-1, rim, Robo, Robo1, Robo3 cytoplasmic, Robo3 extracellular, Rop, Ror2, RORgt, rough Drosophila protein, Rumpelstiltskin, ryanodine receptor (e.g., skeletal), ryanodine receptors, S-adenosylhomocysteine hydrolase, S100 calcium binding protein A2, SAC1, sarcalumenin, SAX7, scabrous Drosophila gene protein, Schwann cell & myoblast plasma membrane glycoprotein, Schwann cell marker (P(o)), Schwann cell myelin protein, E-selectin, Sema I, Sema II, sensory cilia and excretory pore marker, Sex combs reduced protein, sex-lethal protein, Shot, sialomucin complex (Muc4), sidestep, Single-minded, Sir2, SIX5, SMN-interacting protein-1 (gemin2), SMN protein aa 28-91, SMN protein aa 159-209, SMN protein aa 159-209 Exon 4, SMN protein aa 210-241 Exon 5, skeletal muscle marker (102 kDa), slit protein, Slug, Smoothened (Smo), somatomedin-C(Sm-C/IGF-1), sonic hedgehog, spectrin (alpha), spermidine or spermine N1-acetyltransferase 1, Spindle-F (e.g., full length protein), Spitz (e.g., extracellular domain), squamous cell carcinoma antigen 1, Squash protein (CG 4711, e.g., full length protein), Squid A protein (e.g., full length), Squid S protein (e.g., full length), SQV-8, SSEA-1, SSEA-3, SSEA-4, STRO-1, stromal cell line, stromal cell surface marker, Sucrose-isomaltase, Sulfotransferase family 1E (e.g., estrogen-preferring, member 1), SUMO-1, SUMO-2, Suppressor of fused (Su (fu)), sympathoadrenal marker, synapsin, synaptic vesicles, synaptobrevin, synaptotagmin, synaptotagmin (e.g., cytoplasmic domain), syntaxin, synuclein-gamma, talin, talinA (e.g., N-terminal), Talin (e.g., carboxy terminus 534 amino acids), Tannerella forsythia, Tango, tau, TCR alpha/beta, TCR beta, tenascin, tetraspanin (e.g., large extracellular loop amino acids 111-217), TGFb3 (e.g., active domain), TGFb3 (e.g., latent domain), titin, Topoisomerase I, tracheal system, transferrin receptor, transglutaminase (e.g., tissue), transitin, transketolase, triadin, Trio, tropomyosin (e.g., gizzard), tropomyosin (e.g., muscle), tropomyosin (e.g., recombinant, isoform 5 and 4 fusion (hTMS/4) protein), troponin I (e.g., cardiac), troponin I (e.g., skeletal and cardiac), troponin T, troponin T (e.g., cardiac), TRP protein, Truncated version of sry-a protein (e.g., amino acids 46 to 530), TSLP, tubulin, alpha-tubulin, beta-tubulin, tyrosine 3-monooxygenase or tryptophan 5-monooxygenase activation protein epsilon polypeptide, tyrosine hydroxylase, ubiquitin conjugating enzyme E2C, Ultrabithorax protein, UNC-10, Us9 (pseudorabies virus), utrophin, V_H_ATPase c-subunit, vasa, VCAM, VCAM-1, versican (e.g., hyaluronate-binding region), vimentin, vinculin, vinculin (e.g., meta-vinculin), visinin, Wash cDNA/GST fusion protein, Windbeutel protein (e.g., N-terminal half), Wingless protein, wit, wound epithelium & transport/secretory cytoskeletal protein, wound epithelium & transport/secretory cell protein (e.g., 42 kDa), wrapper, Xenopus nuclear factor, xnf7, xenotropic murine leukemia virus-related virus P12, Yan Drosophila protein, zeugmatin, ZO-1, ZwS, ABRA1, AHNAK1, ARAP1, beta-Catenin, GRAP2/GADS, Grb2, LAT, LIME, LST1, NHERF1/EBP50, NTAL/LAB, PAG/Cbp, PRR7/TRAP3, SIT, SLP76, TRIM, EGFR, EGFR (Phospho-Tyr992), EGFR (Phospho-Tyr1173), Fyn, Lck, Lyn, MEK ½, MRCK alpha, PDK1, Phosphotyrosine, PKAc, Placental alkaline phosphatase, PTEN, SHIP-1, Syk, ZAP-70, alpha/beta-tubulin dimer, alpha-tubulin, beta-tubulin, betaIII-tubulin, gamma-tubulin, Kinesin, Kinesin (heavy chain), MAP2ab, Cytokeratin, Cytokeratin 5+18, Cytokeratin 7+17, Cytokeratin 8, Cytokeratin 10, Cytokeratin 10+13, Cytokeratin 18, Cytokeratin 19, GFAP, Lamin C, Neurofilament heavy protein, Neurofilament medium protein, Vimentin, Brg1, CRP, CtBP1, Cyclin D1, Daxx, FoxP3, Hsp90 alpha/beta, Hsp90 beta, Ku80 Antigen/DNA helicase p80, Lamin C, p21Waf1, p53, p53 (Phospho-Ser392), PTEN, STAT1, STAT1 (Phospho-Ser727), TCF4/TF7L2, Ubinuclein 1, AGR2+AGR3, AGR3, Albumin, alpha-Fetoprotein, beta2-microglobulin, Clusterin, CRP, Prostate-specific antigen (PSA), Transferrin, beta Endorphin, Chorionic gonadotropin (beta-hCG), Growth hormone (hGH), Growth hormone+HRP, Progesterone, Thyroglobulin, Thyrotropin (hTSH), IgA, IgA secretory component, IgE, IgG (Fab), IgG (Fc), IgM, Kappa light chains, Lambda light chains, Bcl2, Granzyme B, H-ras, Sos, CPNE7, STIM1, IFN-gamma, SOCS3, Intra-Acrosomal Protein, Placental alkaline phosphatase, Clathrin heavy chain, betaIII-tubulin, CD230/Human Prion Protein (PrP), GCPII/PSMA, GFAP, MAP2ab, Neurofilament heavy protein, Neurofilament medium protein, PRR7/TRAP3, EBV antigen EBNA-1, HBV antigen HBsAg, HBV antigen HBsAg, HIV protease, HIV-1 gp24, HSV1 (gC), HSV1+HSV2 (gB), HSV2 (gG), Mycobacterium tuberculosis antigen CFP10 (Rv3874), Mycobacterium tuberculosis antigen EsaT-6 (Rv3875), Neisseria meningitidis antigen Orf1/FrpD, Horseradish peroxidase (HRP), IgA secretory component, MFG, Myeloperoxidase (MPO) and Tenascin.

### Antigen Delivery

In some aspects, a plurality of antigen species is delivered to a single animal. In specific aspects, a plurality of antigen species is delivered to a single animal, wherein each antigen species is delivered to the animal at an anatomically distinct location. As used herein, the term “antigen species” refers to a first antigen having a feature that differs from a feature of a second antigen. In some cases, the feature that differs is nucleotide sequence, amino acid sequence, secondary, tertiary and/or chemical structure, epitope and/or immunogenicity. In some cases, a first antigen species is a nucleic acid comprising a nucleotide sequence that differs by one nucleotide base or more from the nucleotide sequence of a second antigen species when the nucleotide sequences of the first and second antigen species are aligned. In some cases, a first antigen species is a polypeptide comprising an amino acid sequence that differs by one amino acid or more from the amino acid sequence of a second antigen species when the amino acid sequences of the first and second antigen species are aligned. In some cases, a first antigen species is one type of antigen, (e.g. a polypeptide) and a second antigen species is a different type of antigen (e.g., a nucleic acid). In some cases, a plurality of antigen species comprises different types of antigens, including, but not limited to, polypeptides, polynucleotides, carbohydrates, haptens, and chemicals. For example, a plurality of antigens may include a protein antigen and a carbohydrate antigen. In some cases, a plurality of antigen species comprises different antigens from the same molecule, (e.g., different peptide regions of a single polypeptide). As used herein, the terms “multiple antigens” or “a plurality of antigens” refer to two or more distinct antigen species. In some aspects, two antigen species are delivered to a single animal. In some aspects, two or more antigen species are delivered to a single animal. In some aspects, between two to three antigen species are delivered to a single animal. In some aspects, three antigen species are delivered to a single animal. In some aspects, between two to four antigen species are delivered to a single animal. In some aspects, four antigen species are delivered to a single animal. In some aspects, between two to six antigen species are delivered to a single animal. In some aspects, between two to ten antigen species are delivered to a single animal. For example, in some aspects, 2, 3, 4, 5, 6, 7, 8, 9 or 10 antigen species are delivered to a single animal. In some aspects, more than ten antigen species are delivered to a single animal. In specific aspects, each antigen species is delivered at an anatomically distinct location.

Antigens may be delivered by any delivery route known in the art or a combination of delivery routes. Such delivery routes include, without limitation, intradermal (i.e. into the skin), subcutaneous (i.e. under the skin), intramuscular (i.e. into a muscle), intraperitoneal (i.e. infusion or injection into the peritoneum), intravenous (i.e. into a vein), intranodal (i.e. into a lymph node), intrasplenic (i.e. into the spleen), footpad injection (i.e. combination of intradermal and subcutaneous routes), topical, transdermal (i.e. across the skin), intraductal (i.d.), per os (p.o.; oral; i.e. through the mouth), sublingual (i.e. under the tongue), buccal (i.e. between the cheek and gums), enteral (i.e. through the gastrointestinal tract), topical, nasal, epidural (i.e. injection or infusion into the epidural space), and intravitreal (i.e. through the eye). In some aspects, one or more antigens are injected at anatomically distinct locations. In some aspects, one or more antigens are injected via one or more of the delivery routes above.

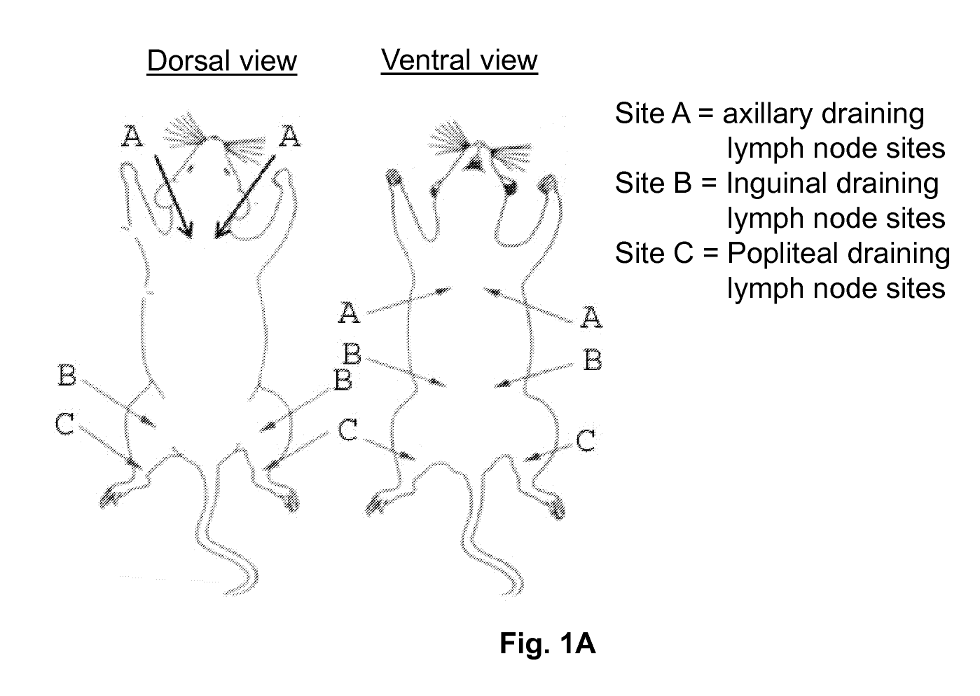

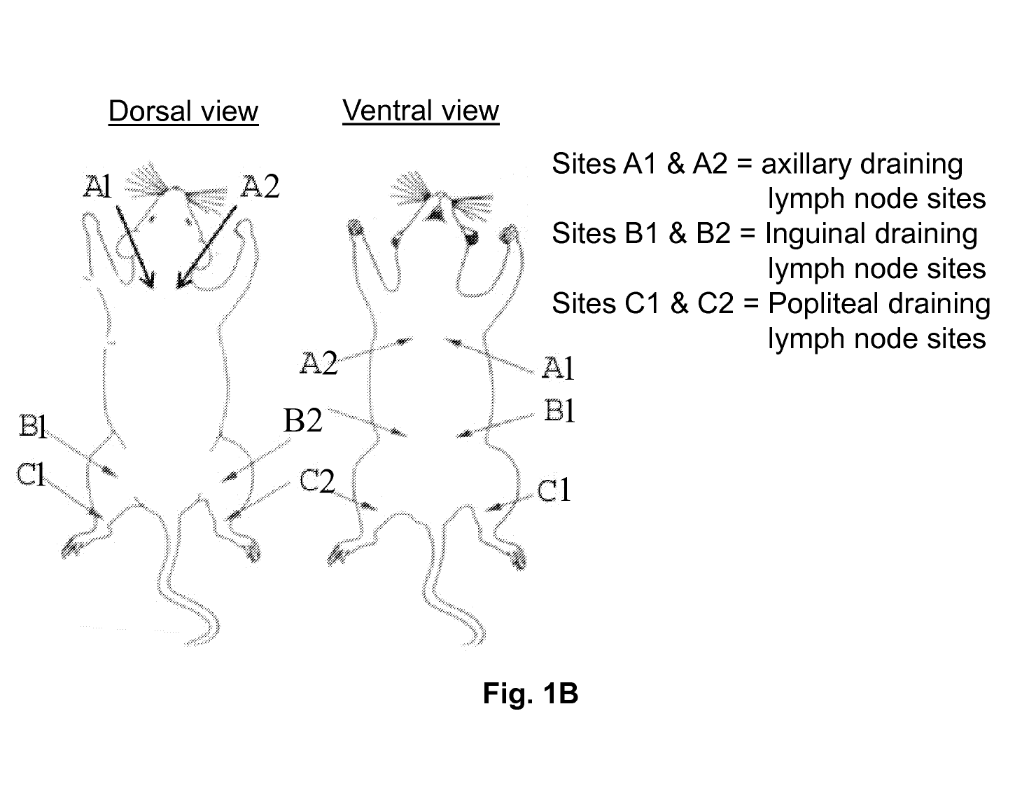

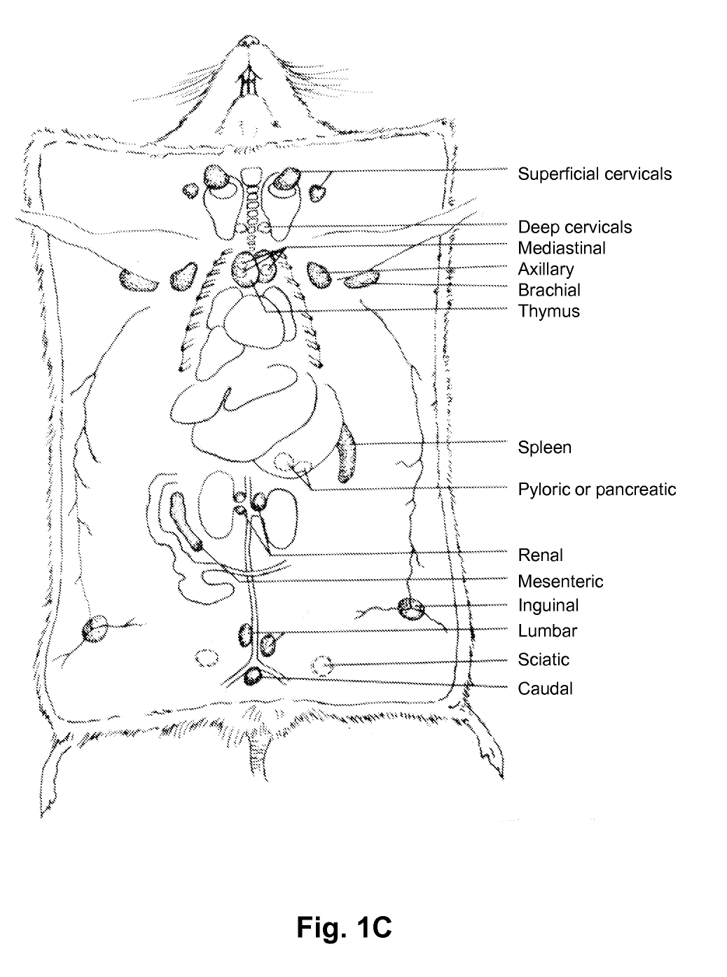

Immune responses in an immunized host animal can sometimes differ, depending on the antigen(s) delivered and/or the delivery route(s) used. Antigens delivered intravenously, for example, typically are routed by the host to the spleen and/or lymph nodes. In some cases, soluble antigens may be delivered intravenously, with or without adjuvant. Adjuvants that may be used intravenously include liposomes, typically prepared as water-in-oil double emulsions and dispersed alum. In some cases, an intravenous route is used for booster injections following primary immunization with an antigen delivered via another route. Antigens delivered intradermally, for example, can be rapidly taken up into the lymphatic system. Typically, the large number of Langerhan's dendritic cells in the dermis transport intact and processed antigen to draining lymph nodes. Antigens delivered subcutaneously, for example, also are typically taken up by the lymphatic system. Rate of absorption can depend on, for example, blood flow in the area, skin temperature, activity of underlying muscles, and contact area. In some cases, areas of loose skin can allow further spread of an antigen preparation, thereby increasing the contact area. Antigens delivered intramuscularly, for example, can be rapidly taken up into the bloodstream and lymphatic system. Absorption properties can depend on, for example, antigen size. Small antigens (e.g., small molecular weight molecules) can be rapidly absorbed into the blood and sometimes can induce a distributed immune response. Large antigens (e.g., high molecular weight molecules) are typically absorbed by the lymphatic system which lies in the fascial planes. Antigens delivered intraperitoneally, for example, can be rapidly taken up by the lymphatic system (e.g., MALT) and transferred to draining lymph nodes, thoracic duct and the vascular system. Relatively large volumes of antigen preparation typically can be delivered, several different types of adjuvants can be used, and the antigen can be widely distributed to lymphoid tissue. Intranodal and intrasplenic delivery routes, for example, allow for direct delivery of antigen to lymphoid tissues. Such routes can be useful when small quantities of antigen are available. FIG. 1C, provides a schematic of the location of specific lymph nodes within the mouse. In certain aspects, each of two or more of the antigens of a plurality of antigen species is delivered intranodally. In one aspect, each of the two or more antigens is delivered intranodally to a different lymph node. In another aspect, each of the two or more antigens is delivered intranodally to more than one lymph node in a particular anatomical location (e.g., the lumbar lymph nodes, see FIG. 1C).

In some aspects, each of two or more of the antigens of a plurality of antigen species is delivered to a single animal at an anatomically distinct location. In some aspects, each antigen species in the plurality of antigen species is delivered at an anatomically distinct location, and no antigen species is delivered at the same location. As used herein, the term “anatomically distinct” location refers to a first location in/on an animal that is physically and/or systemically different than a second location. In some cases, the anatomically distinct locations are physically separated from each other such that the antigens, once delivered, do not compete with one another for the host's immune response. An anatomically distinct location also is the site of antigen species delivery by a human or device, and not necessarily the site in the animal to which an antigen migrates after delivery by the human or device. Such anatomically distinct locations include, without limitation, abdominal cavity, skin, muscle and various organs. In some cases, anatomically distinct locations include locations on the skin that are physically separate from each other, which include, without limitation, foot pad, tail, front leg (left or right), hind leg (left or right), back, abdomen, scruff, head, neck, face, and chest. In certain aspects, anatomically distinct locations include, axillary draining lymph node sites located close to the arm pits of the upper limbs; inguinal draining lymph nodes located close to the mid-section of the animal; and popliteal lymph node draining sites located behind the hind legs (see for example FIG. 1A). In other aspects, anatomically distinct locations include, axillary draining lymph node sites located close to the left arm pits and the right arm pits of the upper limbs; inguinal draining lymph nodes located close to the left side mid-section and the right slide mid-section of the animal; and popliteal lymph node draining sites located behind the left hind leg and the right hind leg (see for example FIG. 1B).

In some aspects, a plurality of antigen species is delivered at the same time or substantially the same time. For example, each antigen species can be delivered simultaneously or sequentially in a single immunization session. In some aspects, each antigen species is delivered at a different time. For example, two or more antigen species can be delivered during separate immunization sessions. Immunization sessions may be minutes apart, hours apart, or days apart. In some cases, immunization sessions can be between 1 to 60 minutes apart. In some cases, immunization sessions can be between 1 to 24 hours apart. In some cases, immunization sessions can be between 1 to 10 days apart.

In some aspects, booster immunizations are administered for one, some or all of the antigen species. In some aspects, booster immunizations are administered for each antigen. In some aspects, booster immunizations are administered for some, but not all, of the antigen species. Boosters for each antigen species can be administered according to any of the dosage, scheduling and/or delivery specifications described below or known in the art. Booster dosage, scheduling and/or delivery specifications can be the same or different for one, some or each antigen species. In some aspects the booster dosage is smaller than that used in the initial immunization. In certain aspects, a subsequent booster immunization dosage is smaller than that used in the prior booster. In some aspects, an animal is boosted with between about ½ to about ⅛ the original amount of antigen. In some aspects, an animal is boosted with between about ⅕ to about 1/10 the original amount of antigen. In some aspects, less than 5 μg of antigen are administered. In certain aspects, less than 2.5 μg of antigen are administered. Particular, immunization and booster dosages are exemplified in the Examples provided herein.

In some aspects, an animal is boosted about one day to about two weeks after the initial immunization. In certain aspects, an animal is boosted about one day to about one week after the initial immunization. For example, an animal can be boosted about 1, 2, 3, 4, 5, 6, or 7, days after the initial immunization. In one aspect, an animal is boosted about two days after the initial immunization. In some cases, the animal is bled after a first booster (e.g., within about 7 to 14 days), and the serum is assayed for antibody titer. In some cases, animals are boosted until the titer plateaus. In some aspects, an animal is boosted between one to ten times. For example, an animal can be boosted 1, 2, 3, 4, 5, 6, 7, 8, 9 or 10 times. In some aspects, an animal can be boosted more than ten times. In one aspect, an animal is boosted between one and seven times. In another aspect an animal is boosted about four to six times. In some aspects, each booster is administered between about one day to about two weeks apart. In one aspect, each booster can be administered about 1, 2, 3, or 4 days apart. In certain aspects, an animal is boosted two to six times and each booster is administered two days apart (i.e. every other day after the initial immunization). In some aspects, a booster can be delivered to the same location as the initial immunization site. Particular, immunization and booster schedules are exemplified in the Examples provided herein.

In some cases, adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and human adjuvants such as BCG (bacille Calmette-Guerin) and Corynebacterium parvum. Additional adjuvents are described in Petrovski and Aguilar, 2004, Immunology and Cell Biology, 82:488-496 and a number of adjuvents are commercially available (e.g., TiterMax®, from Sigma). In some aspects, an adjuvant is used with one or more antigen species.

In some cases, it may be useful to conjugate one, some or each antigen (e.g., when synthetic peptides are used) to a protein that is immunogenic in the species to be immunized. For example, one or more antigens each can be conjugated to keyhole limpet hemocyanin (KLH), serum albumin, bovine thyroglobulin, or soybean trypsin inhibitor, using a bifunctional or derivatizing agent (reactive group), e.g., activated ester (conjugation through cysteine or lysine residues), glutaraldehyde, succinic anhydride, SOCl2, or R1N═C═NR, where R and R1 are different alkyl groups. Conjugates also can be made in recombinant cell culture as fusion proteins. In some aspects, one or more antigen species is conjugated to an immunogenic protein.

These and other conditions under which an animal produces antibodies to an administered antigen known in the art can be used in the methods herein.

### Immunogenic Responses

An immune response often is generated in an animal when conducting methods described herein. In some aspects, the immune response results in the production of antibodies that are specific for the antigen or antigens delivered to the animal. In some aspects, the immune response results in the production of a plurality of antibody species that each specifically bind to a different antigen species. As used herein, the term “antibody species” refers to a first antibody having a feature that differs from a feature of a second antibody. In some cases, the feature that differs is amino acid sequence (e.g., variable region amino acid sequence) and/or antigen specificity. Immune responses resulting in the production of antibodies typically involve certain types of white blood cells (i.e. leukocytes). Lymphocytes, specifically B lymphocytes (B cells), are a type of white blood cell that produces antibodies. B lymphocytes carry antigen-specific receptor molecules that recognize specific targets. Typically, the receptor is an antibody molecule on the B cell surface and each lineage of B cell expresses a different antibody. Such antibody molecules can bind to specific foreign antigens. This antigen/antibody complex is taken up by the B cell and processed by proteolysis into peptides. The B cell then displays these antigenic peptides on its surface major histocompatibility complex (MHC) class II molecules. This combination of MHC and antigen attracts a matching helper T cell, which releases lymphokines and activates the B cell. As the activated B cell then begins to divide, its offspring (i.e. plasma cells) secrete millions of copies of the antibody that recognizes this antigen. These antibodies circulate in blood plasma and lymph, bind to pathogens expressing the antigen and mark them for destruction by complement activation or for uptake and destruction by phagocytes. Antibodies also can neutralize invaders directly, such as by binding to bacterial toxins or by interfering with the receptors that viruses and bacteria use to infect cells.

B cells generally originate from a common lymphoid progenitor and differentiate and develop within the bone marrow. Following maturation, B cells enter the circulation and peripheral lymphoid organs and tissue (e.g. spleen, lymph nodes, and mucosa-associated lymphoid tissue (MALT)) where they reside until needed. The spleen is an organ that has several functions, some of which are involved in the immune system. For example, the spleen houses B cells, as described above, which synthesize antibodies. The spleen also removes antibody-coated bacteria and antibody-coated blood cells by way of blood and lymph node circulation. Lymph nodes are organs of the immune system which typically act as filters or traps for foreign particles. Like the spleen, lymph nodes also house B cells. Lymph nodes are distributed widely throughout the body including the armpit and stomach/gut and linked by lymphatic vessels. Mucosa-associated lymphoid tissue (MALT) (also called mucosa-associated lymphatic tissue) is the diffusion system of small concentrations of lymphoid tissue found in various sites of the body, such as the gastrointestinal tract, thyroid, breast, lung, salivary glands, eye, and skin. MALT typically is involved in regulating mucosal immunity and is populated by lymphocytes such as T cells and B cells, as well as plasma cells and macrophages, each of which is well situated to encounter antigens passing through the mucosal epithelium. The components of MALT are sometimes subdivided into the following: GALT (gut-associated lymphoid tissue); BALT (bronchus-associated lymphoid tissue); and NALT (nose-associated lymphoid tissue).

In some cases, certain responses of the immune system can be spatially segregated and can vary depending on the antigen and/or immunization schedule and/or delivery route, such as the delivery routes described herein. For example, antigens entering the blood (e.g., intravenous delivery) typically are drained into the spleen. In another example, antigens entering the gut or other luminal organs (e.g., intraperitoneal delivery) typically are sensed and acted upon by MALT. In another example, antigens entering the skin (e.g., intradermal delivery) typically are sensed initially by regional lymph nodes including but not limited to lymph nodes at, the axillary draining lymph node sites located close to the arm pits of the upper limbs; the inguinal draining lymph node sites located close to the mid-section of the animal; the popliteal lymph node draining sites located behind the hind legs as described in FIGS. 1A and 1B.

### Monoclonal Antibody Production

Monoclonal antibodies can be prepared using a wide variety of techniques known in the art including the use of hybridoma (Kohler et al., Nature, 256:495 (1975); Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981), recombinant, and phage display technologies, or a combination thereof. Following is a description of non-limiting representative methods for producing monoclonal antibodies which may be used to produce, for example, monoclonal mammalian, chimeric, humanized, human, domain, diabodies, vaccibodies, linear and multispecific antibodies.

Hybridoma Techniques

Methods for producing and screening for specific antibodies using hybridoma technology are routine and well known in the art. In hybridoma methods, a mouse or other appropriate host animal, such as hamster, guinea pig or rat, for example, typically is immunized as described above to elicit lymphocytes that each produce or are capable of producing antibodies that will specifically bind to each antigen used for immunization. At the desired time after immunization, lymphocytes are isolated (e.g. from spleen, lymph nodes, thymus and/or bone marrow) and then fused with an immortal cell (e.g., myeloma cell) using a suitable fusing agent or fusion partner, such as polyethylene glycol, to form a hybridoma cell (Goding, Monoclonal Antibodies: Principles and Practice, pp. 59-103 (Academic Press, 1986)). In the primary antibody response to an antigen the major class of antibody produced is IgM, whereas in the secondary response it is IgG (or IgA or IgE).). In some aspects lymphocytes are isolated after a primary response is elicited. In certain aspects, lymphocytes are isolated 4 to 10 days after an initial immunization. In certain aspects lymphocytes are isolated after a secondary response is elicited. In some aspects, lymphocytes are isolated at least 10 to 14 days after an initial immunization. In certain aspects, lymphocytes are isolated prior to full maturation of the B-cells. In some aspects, lymphocytes are isolated within about 28 days after an initial immunization. It will be understood based on the teachings herein, that one or more boost immunizations may be administered after the initial immunization and prior to lymphocyte isolation.

In certain aspects the lymphocytes are isolated from each distinct location. In one aspect, lymphocytes are isolated from lymph nodes isolated from axillary draining lymph node sites located close to the arm pits of the upper limbs; the inguinal draining lymph node sites located close to the mid-section of the animal; the popliteal lymph node draining sites located behind the hind legs as described in FIG. 1A. In other aspects, anatomically distinct locations include, axillary draining lymph node sites located close to the left arm pits and the right arm pits of the upper limbs; inguinal draining lymph nodes located close to the left side mid-section and the right slide mid-section of the animal; and popliteal lymph node draining sites located behind the left hind leg and the right hind leg (see for example FIG. 1B). In certain aspects, B-cells expressing antigen specific antibodies (antigen specific B-cells) are enriched prior to fusion. Methods for selecting antigen specific B-cells are known in the art (see, for example, Kodituwakku et al. 2003, Immunol & Cell Biol. 81:163-170). In certain aspects, the selected myeloma cells are those that fuse efficiently, support stable high-level production of antibody by the selected antibody-producing cells, and are sensitive to a selective medium that selects against the unfused parental cells. In some aspects, the myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Once hybridoma cells that produce antibodies of the desired specificity, affinity, and/or activity are identified, the clones may be subcloned by limiting dilution procedures and grown (i.e. replicated) in vitro, for example, by standard methods (Goding, Monoclonal Antibodies: Principles and Practice, pp. 59-103 (Academic Press, 1986)). In some cases, the hybridoma cells are replicated in cell culture. Suitable culture media for this purpose include, for example, D-MEM or RPMI-1640 medium. Hybridoma cells also may be grown (i.e. replicated) in vivo as ascites tumors in the abdominal cavity of an animal, for example, by intraperitoneal (i.p.) injection of the hybridoma cells into the animal (e.g., mouse).

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, affinity tags, hydroxylapatite chromatography, gel electrophoresis, dialysis, etc. Examples of purification methods are described in more detail below.

### Antibody Purification and Isolation

In certain aspects, provided are antibodies that are substantially purified and/or isolated. The term “purified” as used herein, refers to a molecule of interest that has been identified and separated and/or recovered from a component of its natural environment. Thus, in some embodiments, an antibody provided herein is a purified antibody where it has been separated from one or more components of its natural environment. For example, a collection of polyclonal antibodies may be purified or substantially purified from its source such as, for example, serum. The term “isolated antibody” as used herein refers to an antibody which is substantially free of other antibody molecules having different structure or antigenic specificities. Thus, in some aspects, antibodies provided are isolated antibodies which have been separated from antibodies with a different specificity. An isolated antibody may be a monoclonal antibody. An isolated antibody that specifically binds to an epitope, isoform or variant of a target may, however, have cross-reactivity to other related antigens, e.g., from other species (e.g., species homologs). An isolated antibody as provided may be substantially free of one or more other cellular materials. In some aspects, a combination of “isolated” monoclonal antibodies is provided, and pertains to antibodies having different specificities and combined in a defined composition. Methods of production and purification/isolation of an antibody are described herein.

Once an antibody molecule has been produced by recombinant, hybridoma, in vivo and/or in vitro methods, it may be purified by any method known in the art for purification of an immunoglobulin molecule, for example, by chromatography (e.g., ion exchange, affinity, particularly by affinity for the specific antigens Protein A or Protein G, and sizing column chromatography), filtration, centrifugation, differential solubility, or by any other standard technique for the purification of proteins. Further, the antibodies herein or fragments thereof may be fused to heterologous polypeptide sequences (referred to herein as “tags”) described above or otherwise known in the art to facilitate purification.

When using in vitro techniques, for example, an antibody can be produced intracellularly, in the periplasmic space, or directly secreted into the medium. If an antibody is produced intracellularly, as a first step, the particulate debris, either host cells or lysed fragments, is removed, for example, by centrifugation or ultrafiltration. Carter et al., Bio/Technology, 10:163-167 (1992) describe a procedure for isolating antibodies which are secreted into the periplasmic space of E. coli. Where the antibody is secreted into the medium, supernatants from such systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

Antibodies prepared from cell culture and/or ascites fluid, for example, can be purified using, for example, hydroxylapatite chromatography, hydrophobic interaction chromatography, ion exchange chromatography, gel electrophoresis, dialysis, and/or affinity chromatography either alone or in combination with other purification steps. The suitability of protein A as an affinity ligand often depends on the species and isotype of the immunoglobulin Fc domain that is present in the antibody. The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX resin (J. T. Baker, Phillipsburg, N.J.) can be useful for purification. Other techniques for protein purification such as fractionation on an ion-exchange column, ethanol precipitation, Reverse Phase HPLC, chromatography on silica, chromatography on heparin, SEPHAROSE chromatography on an anion or cation exchange resin (such as a polyaspartic acid column), chromatofocusing, SDS-PAGE, and ammonium sulfate precipitation are also available depending on the antibody to be recovered. Following any preliminary purification step(s), a mixture comprising an antibody of interest and contaminants may be subjected to low pH hydrophobic interaction chromatography using an elution buffer at a pH between about 2.5-4.5, and performed at low salt concentrations (e.g., from about 0-0.25 M salt).

### Antibody Titer

In some aspects, the titer is measured for each antibody species from an animal immunized with a plurality of antigen species. Antibody titer can be measured by any method known in the art for measuring antibody titer including, but not limited to, solid-phase radioimmunoassay (RIA), direct ELISA, microagglutination techniques, and serological tests (e.g., hemagglutination, complement fixation). In some aspects, the titer for each antibody species from an animal immunized with a plurality of antigen species is substantially equal to the titer for each antibody species from an animal immunized with a single antigen species. As used herein, the term “substantially equal” refers to two titer measurements that differ by about 30% or less. In some aspects, the titer for each antibody species from an animal immunized with a plurality of antigen species is between about 70% to about 100% of the titer for each antibody species from an animal immunized with a single antigen species. For example, the titer for each antibody species from an animal immunized with a plurality of antigen species can be about 70%, 75%, 80%, 85%, 90%, 95%, or 100% of the titer for each antibody species from an animal immunized with a single antigen species.

### EXAMPLES

The examples set forth below illustrate certain embodiments and do not limit the technology.

### Example 1

### Materials and Methods

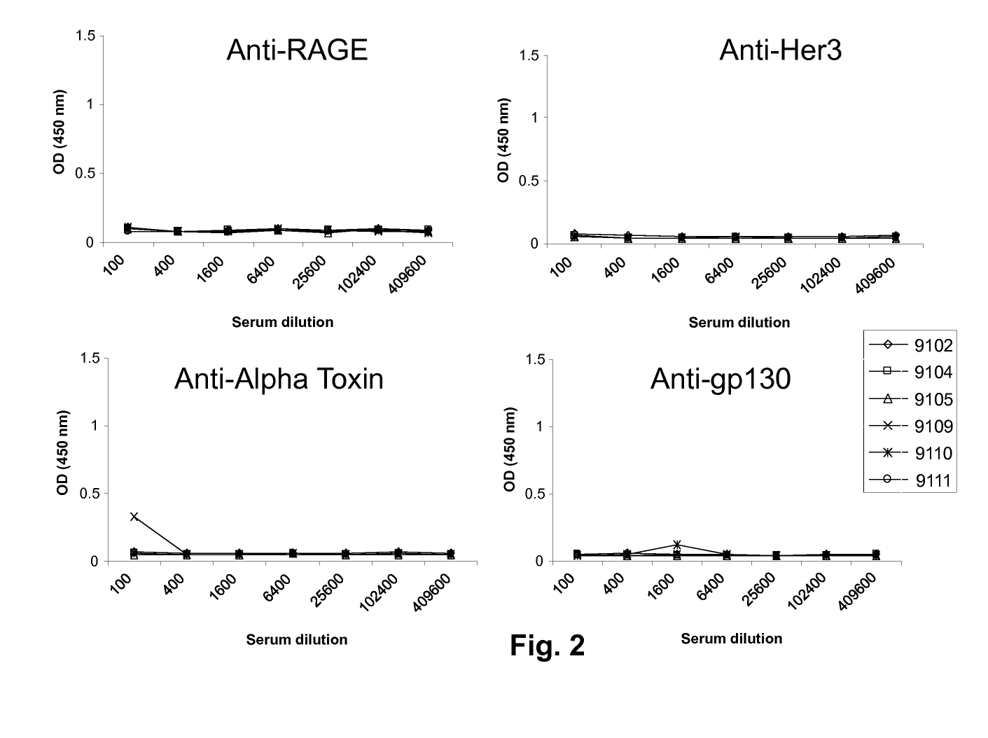

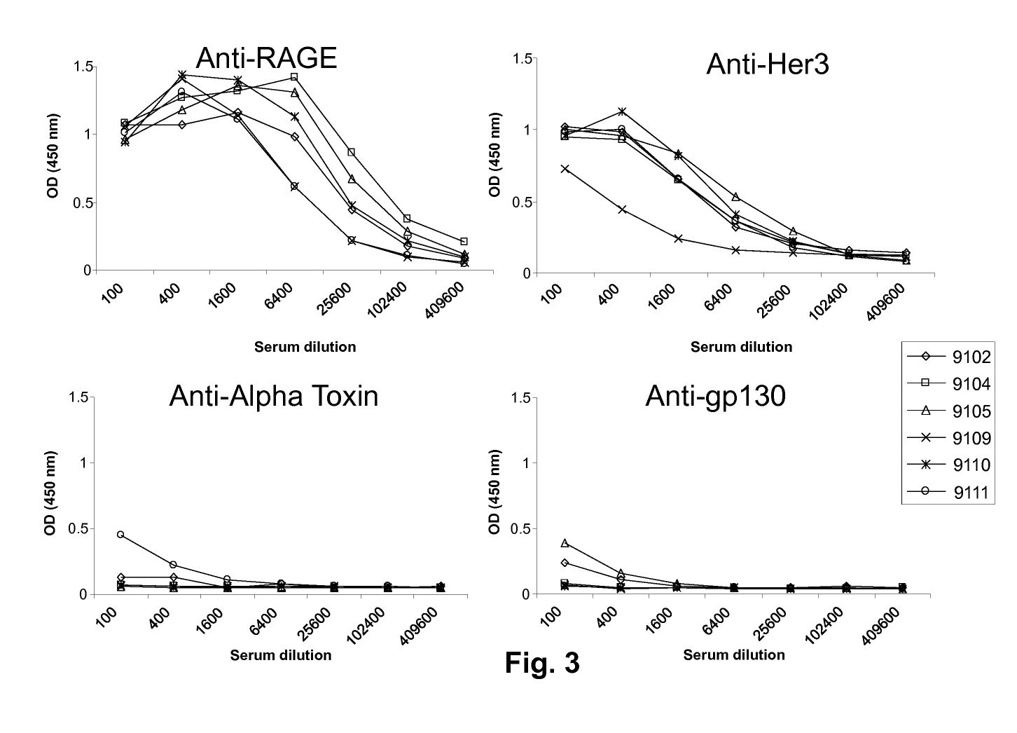

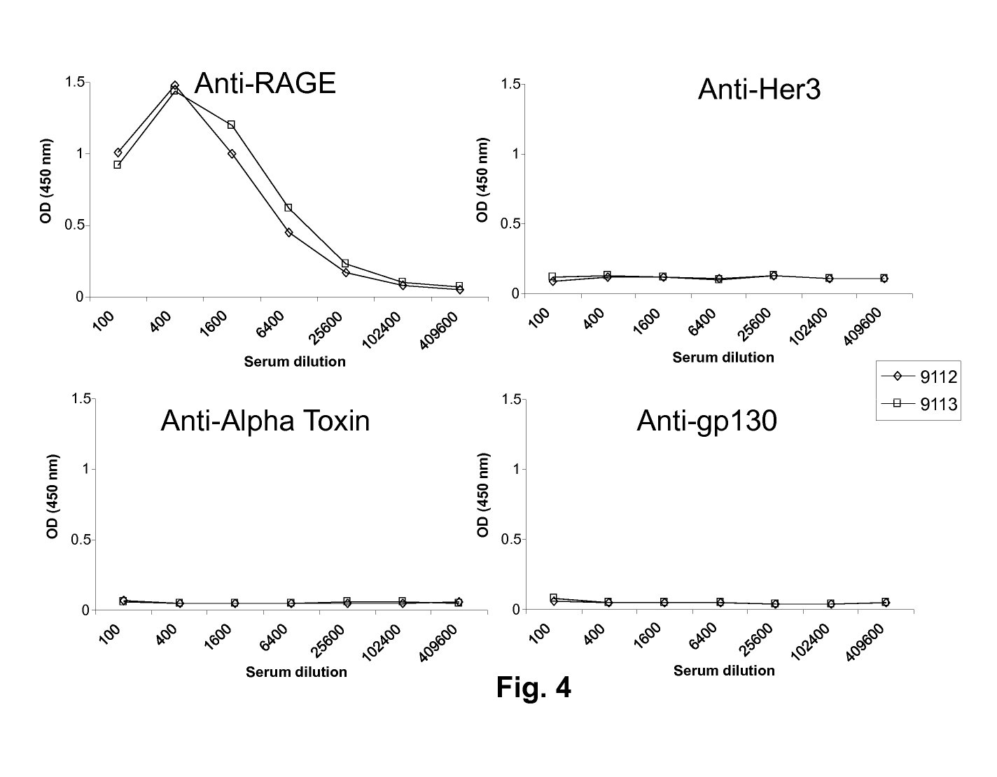

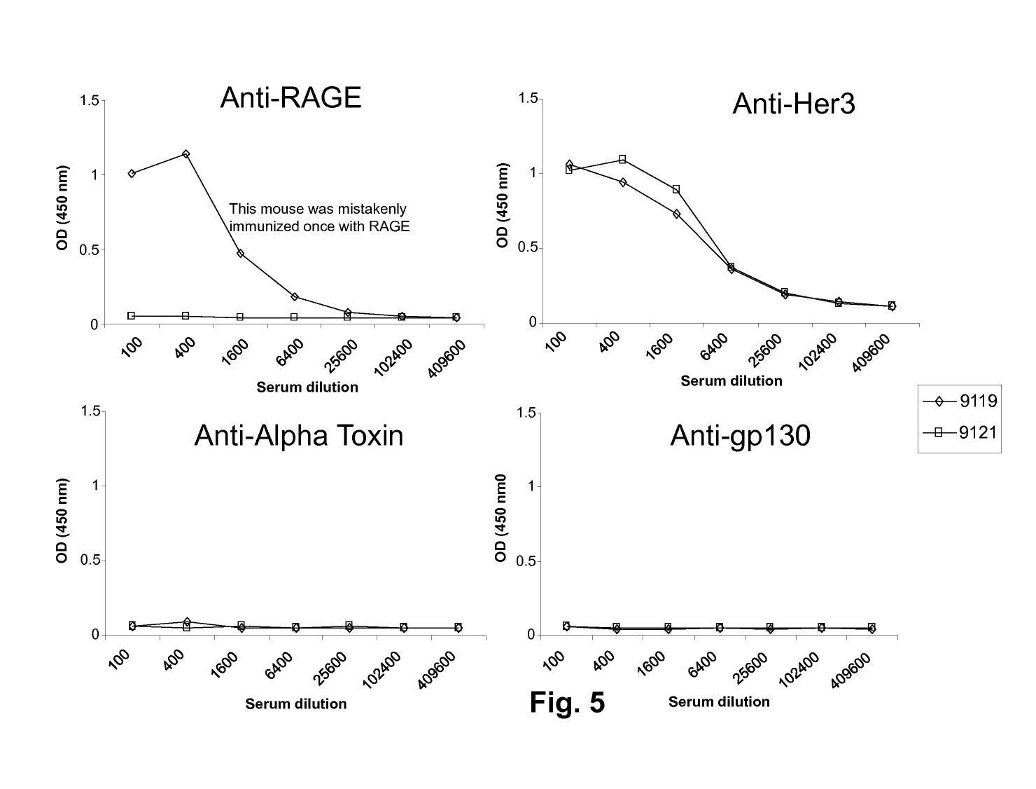

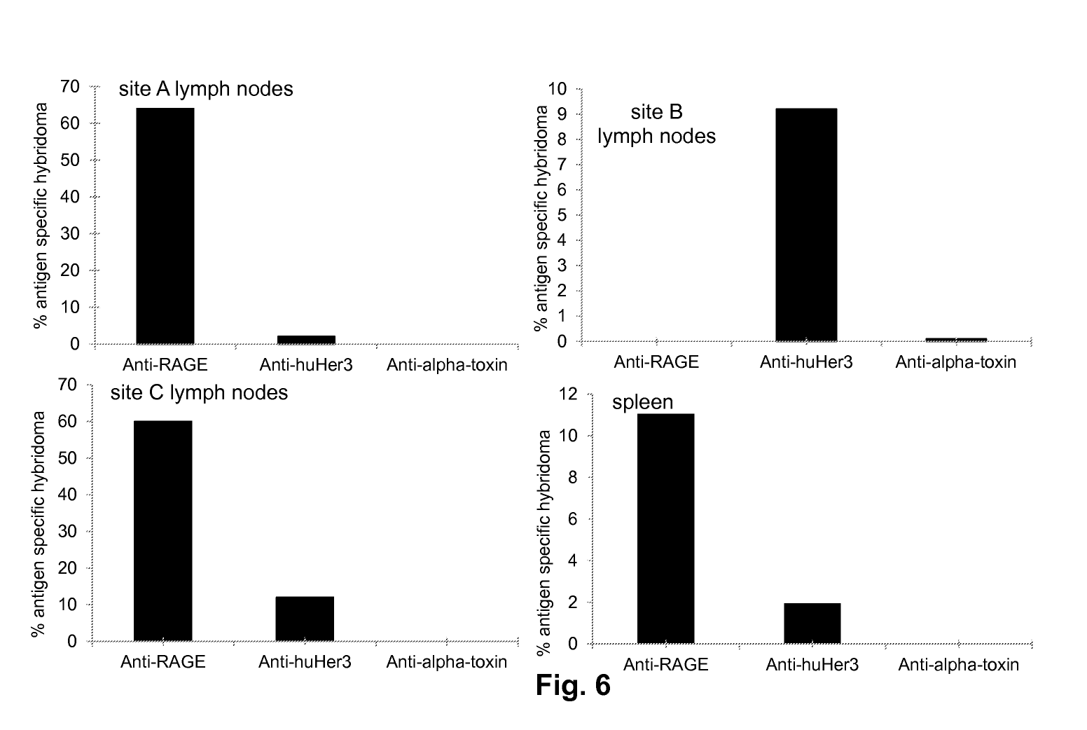

Prior to immunization, pre-bleeds were collected and the titers against huRAGE-his, huHer3-his and α-toxin-his and gp130-his (control antigen) were determined by direct binding ELISA (FIG. 2). Animals were immunized with recombinant huRAGE-his, huHer3-his and α-toxin-his proteins by targeting the each antigen to distinct draining lymph node sites. In this instance, animals were immunized subcutaneously with huRAGE-his, huHer3-his and α-toxin-his closed to the axillary, inguinal and popliteal draining lymph nodes sites, respectively, over the course of 13-day. Each animal was immunized on day 1, 3, 7, 9, 11 and 13. On the scheduled immunization date, each antigen was injected at four sites in one of the three distinct locations (A, B or C) as depicted in FIG. 1A. The amount of each antigen used for each injection was 2.5 mg in 50 μl on day 1, and the amount was subsequently reduced to 1.25 μg/injection in 50 μl on day 3, 0.625 μg/injection in 50 μl on day 7 and 0.3125 μg/injection in 50 μl on day 9, 11 and 13. Throughout the entire immunization process, TiterMax adjuvant (Sigma) was used as an adjuvant. To monitor the immune response against each individual antigen, control mice were immunized with only huRAGE-his, huHer3-his and α-toxin-his at the respective draining lymph node sites as animals received the three antigens. At the end of the 13-day immunization schedule, tested bleeds were collected and the titers against huRAGE-his, huHer3-his and α-toxin-his and gp130-his (control antigen) were determined by direct binding ELISA (FIGS. 3-5). On day 17 post-immunization, splenocytes or lymph node lymphocytes isolated from each distinct drainage sites were fused with P3X myeloma at 1:2 ratio by PEG1500 as described in Antibody manual by Harlow and Lane. The fused cells were then plated out in 96-well plate at 5×10⁴ cells/well in the ExCell-610 supplemented with 10% FBS, 1% Condimed H1 and 1X HAT. Seven days post-fusion, the HAT containing media were removed and the hybridomas were maintained in ExCell-610 supplemented with 10% FBS and 1X HT. The hybridomas derived from each fusion were then screened on day 14 for anti-huRAGE, anti-huHer3 and anti-α-toxin by ELISA (FIG. 6). Separate ELISA plates were coated overnight with 50 μl of a 1 mg/ml of the respective antigens in PBS, pH 7.2. The coating solution were then aspirated out, the plates washed with PBS containing 0.1% Tween-20 (wash buffer) and then blocked with 4% dry milk in wash buffer (blocker) for 1 hour at ambient temperature. The blocker was then removed and serum samples serially diluted 1:2 in blocker was then applied to a series of wells containing the different antigens. After incubation for 1 hour at ambient temperature the plates were washed 3 times with the wash buffer and then rabbit anti-mouse Fcγ specific antibody conjugated to HRP was applied after 1:10,000 dilution in the blocker. After incubation for 1 hour at ambient temperature the plates were washed three times with the wash buffer and three times with PBS. The ELISA wells were then treated with 50 μl of TMB substrate solution for 10 minutes and then the reacts were stopped by adding to each well 50 μl of 1N hydrochloric acid. The color intensity in each well was measured at 450 nm. The color intensity in each well was then plotted as a function of the serum dilution to get the titration curves.

### Results

After 13 day post-immunization, mice (animals 9102, 9104, 9105, 9109, 9110 and 9111) immunized with huRAGE-his, huHer3-his and α-toxin-his show strong anti-huRAGE and anti-huHer3 Ab titer, but no detectable anti-α-toxin titers (FIGS. 3-5). The reason for the different magnitude of IgG titers is likely due to the immunogenicity of each antigen. In addition, the antigen-specific Ab titers for each antigen of animals immunized with multiple antigens is similar to the antigen-specific Ab titer of animals which receive a single antigen. This shows that immunization of an antigen at specific draining lymph node sites does not interfere with the immune system from mounting a humoral response against another unrelated antigen administrated at other draining lymph node sites. The hybridoma screening results (FIG. 6) show that the hybridomas derived from the fusion of lymph node lymphocytes are largely specific for the antigen to which they are exposed even though the same animal is immunized with different antigen at other draining lymph node sites. For instance, hybridomas derived from the huRAGE-immunized axillary draining lymph node sites are overwhelmingly specific for anti-huRAGE. Similarly, majority of the anti-huHer3 hybridomas are derived from fusion of cells isolated from the huHer3-immunized draining lymph nodes. In conclusion, by coupling a site-specific immunization strategy with an ultra-short immunization protocol, it is possible to generate hybridomas specific for multiple antigens in parallel using limited number of animals in a short span of time.

### Example 2

### Materials and Methods

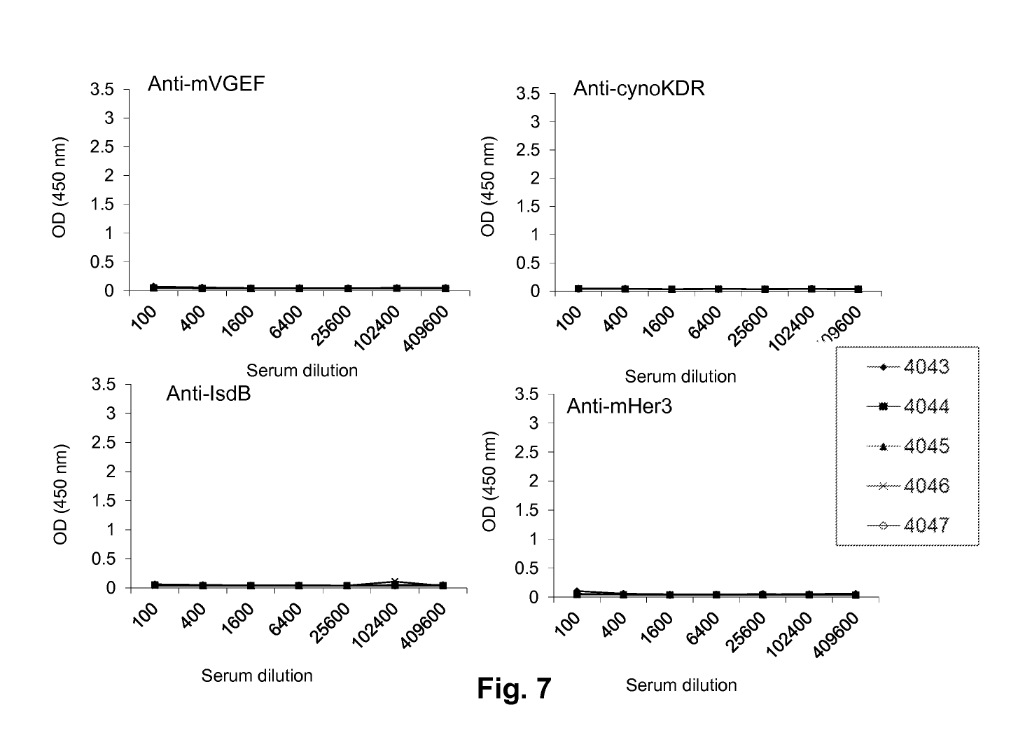

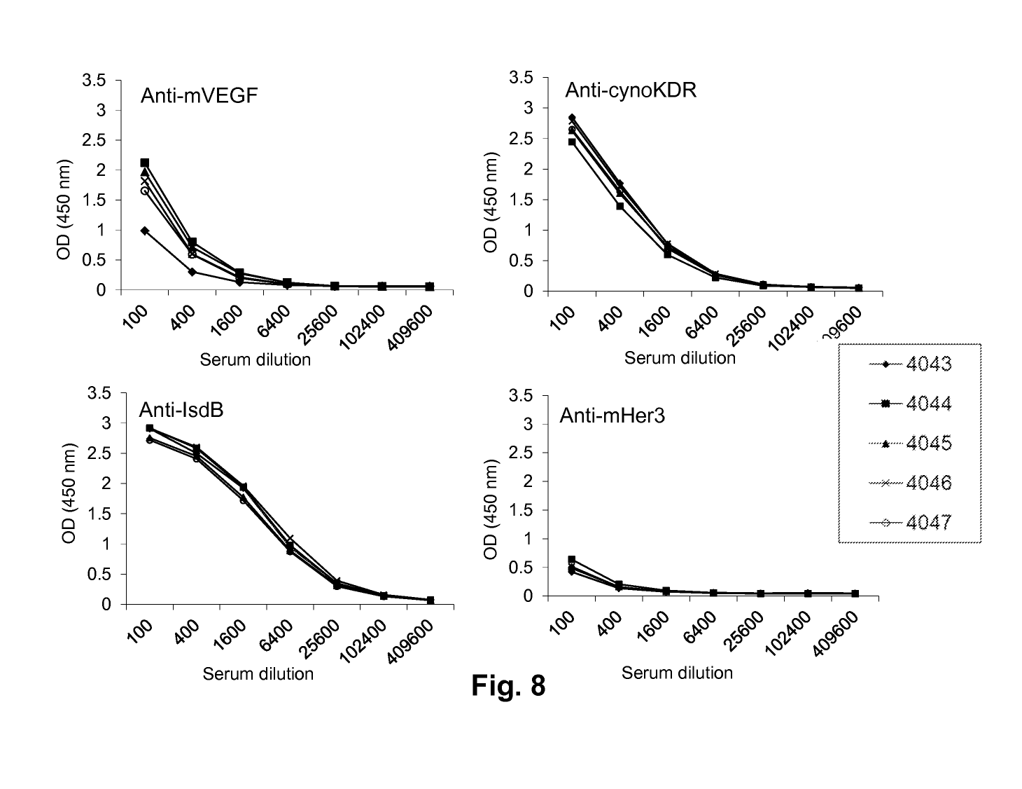

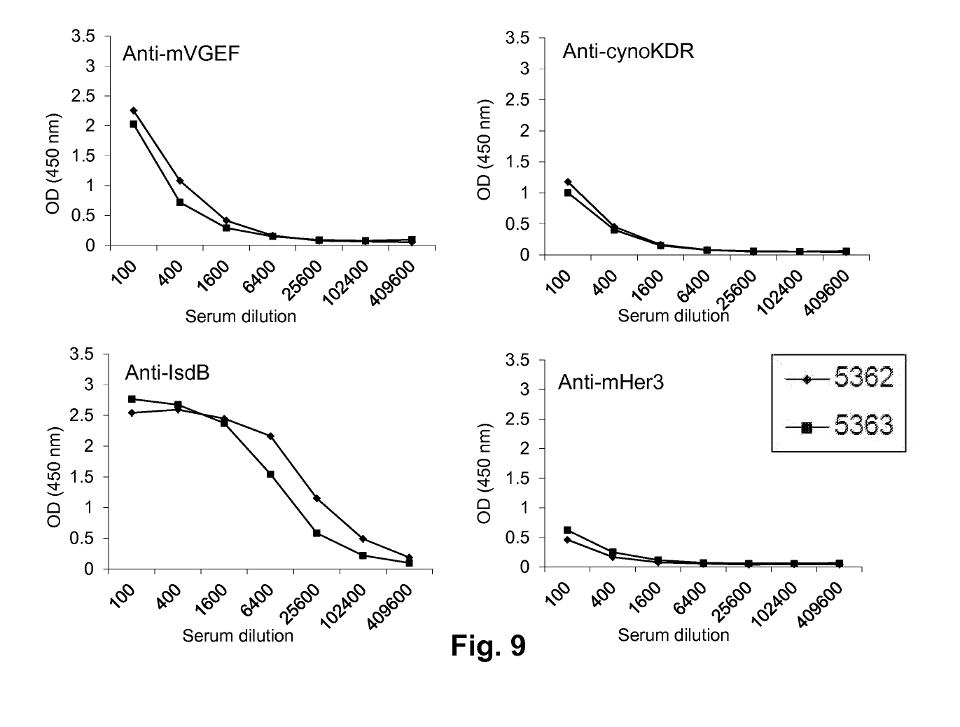

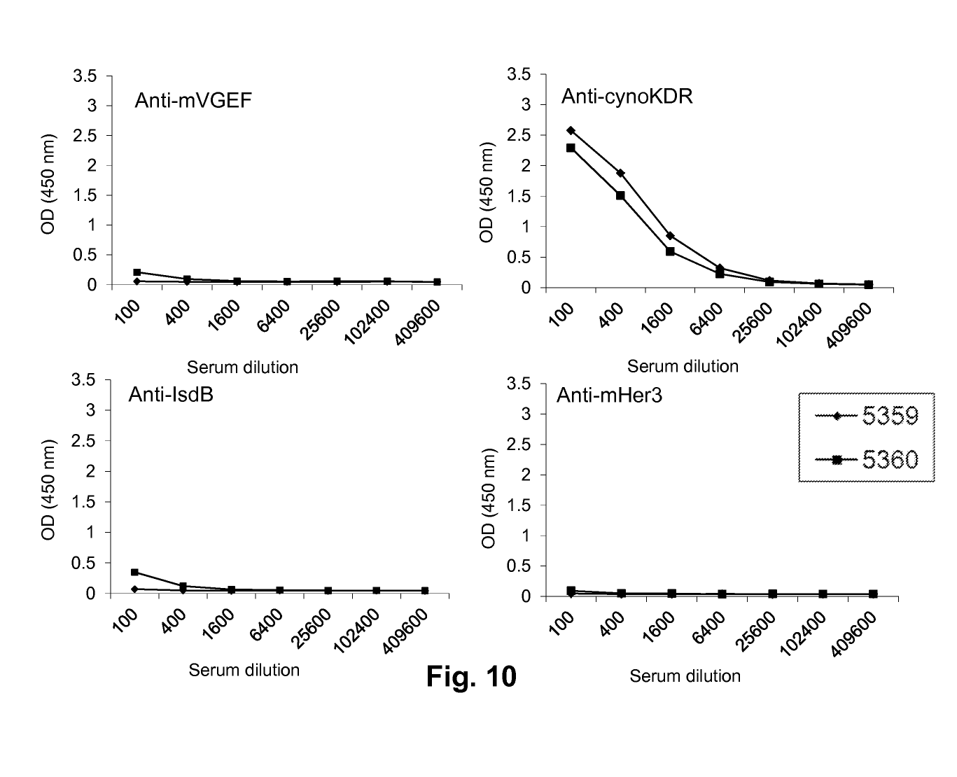

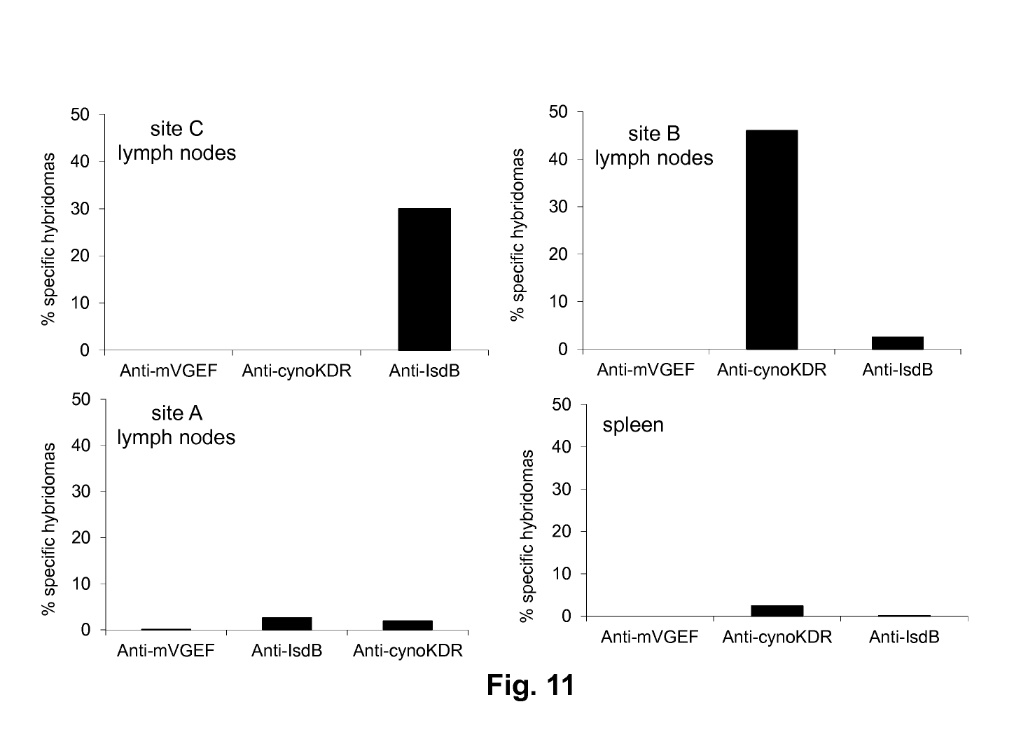

Prior to immunization, pre-bleeds were collected and the titers against mVEGF-his, cynoKDR-his and IsdB-his and mHer3-his (control antigen) were determined by direct binding ELISA (FIG. 7). Animals were immunized with recombinant mVEGF-his, cynoKDR-his and IsdB-his proteins by targeting the each antigen to distinct draining lymph node sites. In this instance, animals were immunized subcutaneously with mVEGF-his, cynoKDR-his and IsdB-his closed to the axillary, inguinal and popliteal draining lymph nodes sites, respectively, over the course of 13-day. Each animal was immunized on day 1, 3, 7, 9, 11 and 13. On the scheduled immunization date, each antigen was injected at four sites in one of the three distinct locations (A, B or C) as depicted in FIG. 1. The amount of each antigen used for each injection was 2.5 μg in 50 μl on day 1, and the amount was subsequently reduced to 1.25 μg/injection in 50 μl on day 3, 0.625 μg/injection in 50 μl on day 7 and 0.3125 μg/injection in 50 μl on day 9, 11 and 13. Throughout the entire immunization process, TiterMax adjuvant (Sigma) was used as an adjuvant. To monitor the immune response against each individual antigen, control mice were immunized with only mVEGF-his, cynoKDR-his or IsdB-his at the respective draining lymph node sites as animals received the three antigens. At the end of the 13-day immunization schedule, tested bleeds were collected and the titers against mVEGF-his, cynoKDR-his and IsdB-his and mHer3-his (control antigen) were determined by direct binding ELISA (FIGS. 8-10). On day 17 post-immunization, splenocytes or lymph node lymphocytes isolated from each distinct drainage sites were fused with P3X myeloma at 1:2 ratio by PEG1500 as described in Antibody manual by Harlow and Lane. The fused cells were then plated out in 96-well plate at 5×10⁴ cells/well in the ExCell-610 supplemented with 10% FBS, 1% Condimed H1 and 1×HAT. Seven days post-fusion, the HAT containing media were removed and the hybridomas were maintained in ExCell-610 supplemented with 10% FBS and 1×HT. The hybridomas derived from each fusion were then screened on day 14 for anti-mVEGF, anti-cynoKDR and anti-IsdB by ELISA (FIG. 11). Separate ELISA plates were coated overnight with 50 μl of a 1 μg/ml of the respective antigens in PBS, pH 7.2. The coating solution were then aspirated out, the plates washed with PBS containing 0.1% Tween-20 (wash buffer) and then blocked with 4% dry milk in wash buffer (blocker) for 1 hour at ambient temperature. The blocker was then removed and serum samples serially diluted 1:2 in blocker was then applied to a series of wells containing the different antigens. After incubation for 1 hour at ambient temperature the plates were washed 3 times with the wash buffer and then rabbit anti-mouse Fcγ specific antibody conjugated to HRP was applied after 1:10,000 dilution in the blocker. After incubation for 1 hour at ambient temperature the plates were washed three times with the wash buffer and three times with PBS. The ELISA wells were then treated with 50 μl of TMB substrate solution for 10 minutes and then the reacts were stopped by adding to each well 50 μl of 1N hydrochloric acid. The color intensity in each well was measured at 450 nm. The color intensity in each well was then plotted as a function of the serum dilution to get the titration curves.

### Results

After 13 day post-immunization, mice (animals 4043, 4044, 4045, 4046 and 4047) immunized with mVEGF-his, cynoDR-his and IsdB-his show strong anti-IsdB Ab titer, followed by anti-cynoKDR and anti-mVEGF titers (FIGS. 8-10). The reason for the different magnitude of IgG titers is likely due to the immunogenicity of each antigen, as IsdB is expected to be the most foreign antigen to the host followed by cynoKDR and mVEGF. In addition, the antigen-specific Ab titers for each antigen of animals immunized with multiple antigens is similar to the antigen-specific Ab titer of animals which receive a single antigen. This shows that immunization of an antigen at specific draining lymph node sites does not interfere with the immune system from mounting a humoral response against another unrelated antigen administrated at other draining lymph node sites. The hybridoma screening results (FIG. 11) show that the hybridomas derived from the fusion of lymph node lymphocytes are largely specific for the antigen to which they are exposed even though the same animal is immunized with different antigen at other draining lymph node sites. For instance, hybridomas derived from the IsdB-immunized popliteal draining lymph node sites are overwhelmingly specific for anti-IsdB. Similarly, majority of the anti-cynoKDR hybridomas are derived from fusion of cells isolated from the cyoKDR-immunized draining lymph nodes. In conclusion, by coupling a site-specific immunization strategy with an ultra-short immunization protocol, it is possible to generate hybridoma specific for multiple antigens in parallel using limited number of animals in a short span of time.

### Example 3

### Examples of Embodiments

A1. A method of producing a plurality of antibody species in a single animal, comprising delivering a plurality of antigen species to a single animal, wherein each antigen species is delivered to the animal at an anatomically distinct location, under conditions in which the animal produces a plurality of antibody species, wherein each antibody species specifically binds to a different antigen species among the plurality of antigen species.

A2. A method of generating an immune response, comprising delivering a plurality of antigen species to a single animal, wherein each antigen species is delivered to the animal at an anatomically distinct location, under conditions in which the animal produces a plurality of antibody species, wherein each antibody species specifically binds to a different antigen species among the plurality of antigen species.

A3. The method of embodiment A1 or A2, wherein the plurality of antigen species are each delivered by a route chosen from intradermal, subcutaneous, intramuscular, intraperitoneal, intravenous, intranodal, intrasplenic, footpad injection, topical, or transdermal.

A4. The method of embodiments A1, A2 or A3, wherein one or more of the antigen species are delivered by injection.

A5. The method of embodiment A4, wherein one or more of the antigen species are injected into the skin.

A6. The method of embodiment A5, wherein one or more antigen species are each injected into the skin at a location chosen from foot pad, tail, front leg, hind leg, back, abdomen, chest, neck, scruff or head.

A7. The method of embodiment A5 or A6, wherein the distinct locations are selected such that the antigen is first sensed by a regional lymph node.

A8. The method of embodiment A7, wherein the distinct locations are selected from those represented in FIG. 1A and/or 1B.

A9. The method of any one of embodiments A1 to A8, wherein one or more antigen species are delivered at substantially the same time.

A10. The method of any one of embodiments A1 to A8, wherein one or more antigen species are delivered at different times.

A11. The method of any one of embodiments A1 to A10, wherein 2 or more antigen species are delivered to the animal.

A12. The method of embodiment A11, wherein 3 or more antigen species are delivered to the animal.

A13. The method of embodiment A12 wherein 4 or more antigen species are delivered to the animal.

A14. The method of embodiment A13, wherein 5 or more antigen species are delivered to the animal.

A15. The method of any one of embodiments A1 to A14, wherein the amount of each antigen species delivered is 2.5 μg or less.

A16. The method of any one of embodiments A1 to A15, wherein the animal is boosted with at least a second delivery of one or more antigen species.

A17. The method of embodiment A16, wherein the animal is boosted with a third delivery of one or more antigen species.

A18. The method of embodiment A17, wherein the animal is boosted with a fourth delivery of one or more antigen species.

A19. The method of embodiment A18, wherein the animal is boosted with a fifth delivery of one or more antigen species.

A20. The method of any one of embodiments A16 to A19, wherein the animal is boosted every other day.

A21. The method of any one of embodiments A16 to A20, wherein the animal is boosted with less of one or more antigen species than was originally delivered.

A22. The method of any one of embodiments A16 to A21, wherein, for each antigen species, the booster is delivered at an anatomical location that is the same as the location of the first antigen delivery.

A23. The method of any one of embodiments A1 to A22, wherein the titer for each antibody species from an animal to which a plurality of antigen species has been delivered is substantially similar to the titer for the antibody species from an animal to which only a single antigen species of the plurality of antigen species has been delivered.

A24. The method of any one of embodiments A1 to A23, wherein the antibodies are isolated.

A25. The method of embodiment A24, wherein the antibodies are isolated from blood, lymph nodes and/or spleen cells

A26. The method of any one of embodiments A1 to A25, further comprising producing one or more hybridoma species.

A27. The method of embodiment A26, wherein one or more hybridoma species are produced using lymph node cells from the animal to which the plurality of antigen species was delivered.

A28. The method of embodiment A27, wherein one or more hybridoma species are produced from regional lymph nodes.

A29. The method of embodiment A28, wherein the regional lymph nodes are selected from those represented in FIG. 1A and/or 1B.

A30. The method of embodiment A26, wherein one or more hybridoma species are produced using spleen cells from the animal to which the plurality of antigen species was delivered.

A31. The method of any one of embodiments A26 to A30, wherein monoclonal antibodies are isolated from the hybridoma.

A32. The method of any one of embodiments A26 to A30, wherein the hybridoma is replicated.

A33. The method of embodiment A32, wherein the hybridoma is replicated in vitro.

A34. The method of embodiment A33, wherein the hybridoma is replicated in cell culture.

A35. The method of embodiment A34, wherein monoclonal antibodies are isolated from the cell culture.

A36. The method of embodiment A32, wherein the hybridoma is replicated in vivo.

A37. The method of embodiment A36, wherein the hybridoma is injected into the abdominal cavity of an animal.

A38. The method of embodiment A37, wherein monoclonal antibodies are isolated from ascites fluid.

A39. The method of any one of embodiments A1 to A38, wherein the animal is a mammal.

A40. The method of embodiment A39, wherein the animal is a rodent.

A41. The method of embodiment A40, wherein the animal is a mouse.

A42. The method of embodiment A40, wherein the animal is a rat.

A43. The method of embodiment A40, wherein the animal is a guinea pig.

A44. The method of embodiment A39, wherein the animal is a rabbit.

A45. The method of embodiment A39, wherein the animal is an ungulate.

B1. A method of producing a plurality of monoclonal antibody species, comprising
a) generating a plurality of hybridoma species from cells from an animal to which a plurality of antigen species has been delivered, wherein each antigen species is delivered to the animal at an anatomically distinct location; and
b) isolating a plurality of monoclonal antibody species from the hybridomas, wherein each antibody species specifically binds to a different antigen species among the plurality of antigen species.

B2. The method of embodiment B1, wherein the plurality of antigen species are each delivered by a route chosen from intradermal, subcutaneous, intramuscular, intraperitoneal, intravenous, intranodal, intrasplenic, footpad injection, topical, or transdermal.

B3. The method of embodiments B1 or B2, wherein one or more of the antigen species are delivered by injection.

B4. The method of embodiment B3, wherein one or more of the antigen species are injected into the skin.

B5. The method of embodiment B4, wherein one or more antigen species are each injected into the skin at a location chosen from foot pad, tail, front leg, hind leg, back, abdomen, chest, neck, scruff or head.

B6. The method of embodiment B4 or B5, wherein the distinct locations are selected such that the antigen is first sensed by a regional lymph node.

B7. The method of embodiment B6, wherein the distinct locations are selected from those represented in FIG. 1A and/or 1B.

B8. The method of any one of embodiments B1 to B7, wherein one or more antigen species are delivered at substantially the same time.

B9. The method of any one of embodiments B1 to B7, wherein one or more antigen species are delivered at different times.

B10. The method of any one of embodiments B1 to B9, wherein 2 or more antigen species are delivered to the animal.

B11. The method of embodiment B10, wherein 3 or more antigen species are delivered to the animal.

B12. The method of embodiment B11, wherein 4 or more antigen species are delivered to the animal.

B13. The method of embodiment B12, wherein 5 or more antigen species are delivered to the animal.

B14. The method of any one of embodiments B1 to B13, wherein the amount of each antigen species delivered is 2.5 μg or less.

B15. The method of any one of embodiments B1 to B14, wherein the animal is boosted with a second delivery of one or more antigen species.

B16. The method of embodiment B15, wherein the animal is boosted with a third delivery of one or more antigen species.

B17. The method of embodiment B16, wherein the animal is boosted with a fourth delivery of one or more antigen species.

B18. The method of embodiment B17, wherein the animal is boosted with a fifth delivery of one or more antigen species.

B19. The method of any one of embodiments B15 to B18, wherein the animal is boosted every other day.

B20. The method of any one of embodiments B15 to B19, wherein the animal is boosted with less of one or more antigen species than was originally delivered.

B21. The method of any one of embodiments B15 to B20, wherein, for each antigen species, the booster is delivered at an anatomical location that is the same as the location of the first antigen delivery.

B22. The method of any one of embodiments B1 to B21, wherein the titer for each antibody species from an animal to which a plurality of antigen species has been delivered is substantially similar to the titer for the antibody species from an animal to which only a single antigen species of the plurality of antigen species has been delivered.

B23. The method any one of embodiments B1 to B22, wherein one or more hybridoma species are produced using lymph node cells from the animal to which the plurality of antigen species was delivered.

B24. The method of embodiment B23, wherein one or more hybridoma species are produced from regional lymph nodes.

B25. The method of embodiment B24, wherein the regional lymph nodes are selected from those represented in FIG. 1A and/or 1B.

B26. The method any one of embodiments B1 to B18, wherein one or more hybridoma species are produced using spleen cells from the animal to which the plurality of antigen species was delivered.

B27. The method of any of embodiments B1 to B26, wherein the hybridoma is replicated.

B28. The method of embodiment B27, wherein the hybridoma is replicated in vitro.

B29. The method of embodiment B28, wherein the hybridoma is replicated in cell culture.

B30. The method of embodiment B29, wherein monoclonal antibodies are isolated from the cell culture.

B31. The method of embodiment B27, wherein the hybridoma is replicated in vivo.

B32. The method of embodiment B31, wherein the hybridoma is injected into the abdominal cavity of an animal.

B33. The method of embodiment B32, wherein monoclonal antibodies are isolated from ascites fluid.

B34. The method of any one of embodiments B1 to B33, wherein the animal is a mammal.

B35. The method of embodiment B34, wherein the animal is a rodent.

B36. The method of embodiment B35, wherein the animal is a mouse.

B37 The method of embodiment B35, wherein the animal is a rat.

B38. The method of embodiment B35, wherein the animal is a guinea pig.

B39. The method of embodiment B34, wherein the animal is a rabbit.

B40. The method of embodiment B34, wherein the animal is an ungulate.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

What is claimed is:

1. A method of producing a plurality of antibody species in a single animal, comprising delivering a plurality of antigen species to a single animal, wherein each antigen species is delivered to the animal at an anatomically distinct location, under conditions in which the animal produces a plurality of antibody species, wherein each antibody species specifically binds to a different antigen species among the plurality of antigen species.

2. A method of generating an immune response, comprising delivering a plurality of antigen species to a single animal, wherein each antigen species is delivered to the animal at an anatomically distinct location, under conditions in which the animal produces a plurality of antibody species, wherein each antibody species specifically binds to a different antigen species among the plurality of antigen species.

3. A method of producing a plurality of monoclonal antibody species, comprising:
a) generating a plurality of hybridoma species from cells from an animal to which a plurality of antigen species has been delivered, wherein each antigen species is delivered to the animal at an anatomically distinct location; and
b) isolating a plurality of monoclonal antibody species from the hybridomas, wherein each antibody species specifically binds to a different antigen species among the plurality of antigen species.

4. The method of claim 1 wherein one or more of the antigen species are injected into the skin.

5. The method of claim 4, wherein one or more antigen species are each injected into the skin at a location chosen from foot pad, tail, front leg, hind leg, back, abdomen, chest, neck, scruff or head.

6. The method of claim 5, wherein the distinct locations are selected such that the antigen is first sensed by a regional lymph node.

7. The method of claim 6, wherein the distinct locations are selected from those represented in FIG. 1A and/or 1B.

8. The method of claim 1, wherein one or more antigen species are delivered at substantially the same time.

9. The method of claim 1, wherein one or more antigen species are delivered at different times.

10. The method of claim 1, wherein 2 or more antigen species are delivered to the animal.

11. The method of claim 1, wherein the amount of each antigen species delivered is 2.5 μg or less.

12. The method of claim 1, wherein the animal is boosted with at least an additional delivery of one or more antigen species.

13. The method of claim 12, wherein the animal is boosted with five additional deliveries of one or more antigen species.

14. The method of claim 12, wherein the animal is boosted every other day.

15. The method of claim 12, wherein the animal is boosted with less of one or more antigen species than was originally delivered.

16. The method of claim 12, wherein for each antigen species, the booster is delivered at an anatomical location that is the same as the location of the first antigen delivery.

17. The method of claim 1, wherein the titer for each antibody species from an animal to which a plurality of antigen species has been delivered is substantially similar to the titer for the antibody species from an animal to which only a single antigen species of the plurality of antigen species has been delivered.

18. The method of claim 1, wherein the antibodies are isolated.

19. The method of claim 1, further comprising producing one or more hybridoma species from regional lymph nodes.
